(19) United States
(12) Patent Application Publication
(10) Pub. No.: US 2025/0114168 A1
(43) Pub. Date: Apr. 10, 2025

(54) DENTAL TREATMENT PLANNING

(71) Applicant: Align Technology, Inc., San Jose, CA (US)

(72) Inventors: Aleksandr KALIUZHIN, Yerevan (AM); Sergei IVANOV, Yerevan (AM); Roman BUSHMANOV, Cary, NC (US); Konstantin STOLYAROV, Berdsk (RU); Nikita DOLGOV, Novosibirsk (RU); Ivan AFANASEV, Novosibirsk (RU)

(21) Appl. No.: 18/907,409

(22) Filed: Oct. 4, 2024

Related U.S. Application Data
(60) Provisional application No. 63/587,999, filed on Oct. 4, 2023.
(60) Provisional application No. 63/606,067, filed on Dec. 4, 2023.

(51) Int. Cl. A61C 7/00; A61C 7/08; G16H 20/40; G16H 50/50

(52) U.S. Cl. CPC A61C 7/002; A61C 7/08; G16H 20/40; G16H 50/50

(57) ABSTRACT

Methods and apparatuses (including software) for determining and/or locating dental ridgelines may include identifying patterns corresponding to two or more ridgeline point locations for individual teeth in the 3D model of the patient's dentition and determining a ridgeline for the teeth based on identified patterns corresponding to the two or more ridgeline point locations. The determined dental ridgelines can be used to determine a dental treatment plan. Also described herein are methods and apparatuses for identifying and quantifying round trip tooth movements in a treatment plan. The round trip tooth movements may be used as a basis for adjusting the treatment plan by reducing the amount of round trip movement or eliminating the round trip movement from the treatment plan.

### CLAIM OF PRIORITY

This patent application claims priority to U.S. provisional patent application No. 63/587,999, titled “AUTOMATED RIDGELINE DETECTION FOR TREATMENT PLANNING,” filed on Oct. 4, 2023, and U.S. provisional patent application No. 63/606,067, titled “METHODS AND SYSTEMS FOR INTELLIGENT STAGING AND DIGITAL TREATMENT PLANNING,” filed on Dec. 4, 2023, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Orthodontic and dental treatments using a series of patient-removable appliances (e.g., “aligners”) are very useful for treating a variety of patients. Treatment planning is typically performed in conjunction with the dental professional (e.g., dentist, orthodontist, dental technician, etc.), by manipulating a model of the patient's teeth from an initial configuration (initial tooth positions) to a final configuration (final tooth positions) and then dividing the treatment into a number of intermediate stages (steps). These steps may correspond to individual appliances that may be worn sequentially, with or without additional interventions (e.g., interproximal reductions, extractions, etc.). Treatment planning can include estimating a patient's teeth in a final configuration. Treatment planning can also involve defining intermediate stages corresponding to a person's dentition between an initial arrangement and a target arrangement. This process may be interactive. This process can involve adjusting staging, a target position, etc. based on various factors. Once the treatment plan is finalized, a series of aligners may be manufactured corresponding to the treatment plan; for example, dental appliances (e.g., a series of aligners, a series of palatal expanders, brackets, and wires, etc.) may be obtained and/or manufactured to implement the one or more stages.

In practice, a patient's final tooth positions may be heavily dependent on the patient's initial tooth positions. Characteristics of the patient's teeth may be used to model the patient's teeth and in particular may be used to generate a treatment plan including a target final set of tooth positions as well as one or more intermediate positions. Examples of tooth characteristics may include ridgelines and tooth axes. During the course of treatment, the patient's teeth may be subject to motion on many axes, including tooth rotation. Tooth rotation may make the determination of an accurate initial position of the patient's teeth difficult and prone to error. Furthermore, irregularly shaped teeth may have unusually shaped ridgelines which may cause conventional algorithmic approaches that use ridgelines to provide erroncous tooth positions.

One of the technical challenges of treatment planning relates to dealing with potential collisions that could occur when teeth are moved in various directions. For example, an issue can arise when a tooth is positioned in a corrective movement path of another tooth. Redirecting teeth and/or staggering movements to avoid collisions to deal with these issues may not be optimal and may increase the treatment time.

### SUMMARY OF THE DISCLOSURE

Described herein are methods and apparatuses for determining dental ridgelines, particularly using trained neural networks. The methods and apparatuses (e.g., devices and/or systems, including software and dental appliances) may be used for accurately determining dental ridgelines from dental scans. The ridgelines can be used to determine a patient's tooth positions. In turn, the tooth positions may be used for dental treatment planning and in determining a predicted final position of a patient's teeth upon completion of a dental treatment.

As described herein, a neural network may be trained with labeled images (and/or 3D point clouds) that include dental ridgelines, particularly showing dental ridgelines on irregularly shaped teeth. Irregularly shaped teeth can include chipped teeth, broken teeth, and teeth that may be partially occluded by gingiva. In some cases the neural network may be trained using a supervised learning approach on labeled 3D models of jaw, e.g., on cloud point files containing segmented teeth shapes. In general, irregular teeth may be manually identified; the task of ridgelines detection may be independent of the identification of irregular teeth. I'm in process of developing a detector of irregular teeth now, we plan to use it in ridgelines detection task to detect of irregular teeth before using the model on a case (and for a several other purposes).

The methods and apparatuses described herein may include using a combination of a neural network and a more conventional algorithmic approach to determine dental ridgelines. In some examples, a determination is made on a tooth-by-tooth basis. If a tooth has a regular shape, then the conventional algorithmic approach is used. If a tooth has an irregular shape, then a neural network is used.

Any of the methods described herein may be used for digital treatment planning. In some examples, a method of digital treatment planning can include acquiring a three-dimensional (3D) scan data of a patient's dentition, converting the 3D scan data into a 3D model of the patient's dentition, segmenting, with a neural network, two or more ridgeline point locations for each individual tooth in the 3D model of the patient's detention, and determining a ridgeline for each individual tooth in the patient's dentition based on the two or more ridgeline point locations.

In some examples, the neural network can determine three ridgeline point locations, such as mesial, middle, and distal point locations. The dental ridgeline can be predicted from these ridgeline point locations.

As described herein, the neural network can include two classifiers. A first classifier can determine probabilities of teeth and gingiva locations for the 3D model. A second classifier can determine probabilities of two or more ridgeline point locations from the 3D model. Alternatively, in any of these examples the neural network may be configured to use a single classifier. For example, one neural network may be used which can segment jaw scans and create jaw model with known tooth type; this segmented 3D model (with known tooth type) may be labeled with ridgeline points for use with a neural network.

The probabilities from the neural network can be used to identify locations from the most probable locations. For example, the probabilities of teeth and gingiva locations can be used to locate and/or identify teeth and gingiva locations. In a similar manner, probabilities of mesial, middle, and distal ridgeline point locations can be used to locate and/or identify mesial, middle, and distal ridgeline point locations.

In some examples, the 3D model can include a point cloud representation of the patient's dentition. The point cloud data may come from an intraoral scanner or other similar device.

As described herein, determining the two or more ridgeline point locations may include transforming the point cloud representation into triangle. In some cases, when a predetermined number of triangles share a common vertex, the common vertex may be a ridgeline point.

Any of the methods described herein can determine a treatment plan based at least in part on the determined ridgelines of each individual tooth in the patient's dentition. In some examples, determining the treatment plan can include determining the final position of each tooth in the patient's dentition.

Also described herein is a non-transitory computer-readable medium including instructions that, when executed by one or more processors of a device, cause the device to perform operations comprising acquiring three-dimensional (3D) scan data of a patient's dentition, converting the 3D scan data into a 3D model of the patient's dentition, segmenting the 3D model of the patient's dentition into individual teeth, determining, with a neural network, two or more ridgeline point locations for each individual tooth in the 3D model of the patient's dentition, and determining a ridgeline for each individual tooth in the patient's dentition based on the two or more ridgeline point locations.

In some examples, a method of digital treatment planning can include converting a patient's 3D dental scan data into a 3D model of the patient's dentition, segmenting the 3D model of the patient's dentition into individual teeth, determining whether one or more of the individual teeth include irregular shape characteristics, and determining a ridgeline for each individual tooth in the patient's dentition based on whether one or more of the individual teeth include irregular shape characteristics.

Any of the methods described herein for determining the ridgeline for each individual tooth can include determining, with a trained neural network, two or more ridgeline point locations for each individual tooth in the 3D model of the patient's dentition when the tooth has an irregular shape characteristic.

In some examples, the two or more ridgeline point locations include a mesial, middle, and distal ridgeline point locations.

As described herein, the neural networks can be trained to classify point locations associated with the patient's dentition. In some variations, the neural network can include two classifiers that function on point cloud 3D models of the patient's dentition. One of the classifiers can determine probabilities of the two or more ridgeline point locations from the 3D model. Another classifier can determine probabilities of teeth and gingiva locations from the 3D model.

In general, the neural networks can be trained. Training the neural networks can include a supervised training that uses a plurality of labeled training images. In some variations, the training images can include 3D models labeled with tooth ridgelines. In some cases, determining the ridgeline for each individual tooth can include algorithmically determining a ridgeline when a tooth does not have an irregular shape characteristic.

Described herein is a non-transitory computer-readable medium including instructions that, when executed by one or more processors of a device, cause the device to perform operations comprising converting a patient's 3D dental scan data into a 3D model of the patient's dentition, segmenting the 3D model of the patient's dentition into individual teeth, determining whether one or more of the individual teeth include irregular shape characteristics, and determining a ridgeline for each individual tooth in the patient's dentition based on whether one or more of the individual teeth include irregular shape characteristics.

Also described herein are apparatuses and/or methods that can identify and/or quantify tooth movements in a treatment plan. In some cases, treatment plan may be adjusted to remove or modify one or more of the identified tooth movements.

The methods and apparatuses described herein relate to the field of digital treatment planning, and more particularly to complex computer systems to manage round trip paths of movement of one or more teeth in a treatment plan for correcting the position of one or more teeth. Thus, described herein are methods and apparatuses (e.g., systems and devices) for identifying and quantifying certain tooth movements in a treatment plan. The methods may involve identifying “round trip” movement of a tooth, which, as used herein, can involve positioning a tooth in one direction and then positioning the tooth back toward the opposite direction. Round trip movements may be used to move the tooth out of the way of a tooth path of another tooth. The extent to which the tooth travels may be quantified as a round trip value, which, in some implementations, is calculated based on evaluating movement of the tooth on a stage-by-stage basis over the treatment plan. The roundtrip value of a tooth may be used as a basis to adjust a treatment plan. For instance, a round trip value can be used to reduce an amount of round trip movement and/or eliminate a round trip movement of a tooth altogether.

According to one example, a method of orthodontically treating a patient's teeth includes: receiving a treatment plan for treating the patient's teeth. A treatment plan can include a series of stages for sequentially moving positions of the patient's teeth from an initial arrangement to a target arrangement; determining a round trip value for at least one tooth of the patient's teeth over a course of the treatment plan, wherein the round trip value is a measure of an extent to which the at least one tooth moves in a second direction with respect to a first direction during the treatment plan, wherein the first direction is opposite the second direction; and outputting the round trip value for the at least one tooth.

The method may further include: identifying a peak stage for the at least one tooth over the treatment plan, wherein the peak stage corresponds to a stage in the series of stages that contributes most to the round trip value; and outputting the peak stage for the at least one tooth. The method may further include eliminating or reducing round trip movement of the at least one tooth from the treatment plan. The method may further include calculating a new treatment plan with the eliminated or reduced round trip movement of the at least one tooth. Determining the round trip value may include calculating relative movement distances of the tooth between at least some of the stages of the series of stages, and summing the relative movement distances over the treatment plan, wherein at least one of the relative movement distances has a negative value and at least one of the relative movement distances has a positive value. Determining the round trip value may include calculating a sum of movements in a positive direction greater than a planned movement, wherein the planned movement is a movement from an initial stage to a last tooth key stage of the series of stages, wherein a key stage corresponds to a stage in the series of stages where the tooth has a predetermined position Determining the round trip value may include calculating horizontal movements of the tooth, wherein the horizontal movements include buccal-lingual translations and mesial-distal translations. Determining the round trip value may include calculating one or more of: buccal-lingual translation, mesial-distal translation, extrusion-intrusion translation, mesial-distal rotation, mesial-distal angulation, proclination, and retroclination. Outputting the round trip value may include causing a user interface to display the round trip value. The method may further include: determining a basis for evaluating movement of the at least one tooth over the treatment plan, wherein the movement of the at least one tooth is calculated with respect to an origin of the at least one tooth as a basis; and calculating extrusion-intrusion translational movements of the at least one tooth between the stages of the treatment plan using the origin as the basis. Each stage of the treatment plan may be associated with a corresponding dental appliance configured to reposition the patient's teeth according to the corresponding stage of the treatment plan.

According to another example, a non-transient, computer-readable medium contains program instructions to cause a processor to: receive a treatment plan for treating a patient's teeth, the treatment plan including a series of stages for sequentially moving positions of the patient's teeth from an initial arrangement to a target arrangement; determine a round trip value for at least one tooth of the patient's teeth over a course of the treatment plan, wherein the round trip value is a measure of an extent to which the at least one tooth moves in a second direction with respect to a first direction during the treatment plan, wherein the first direction is opposite the second direction; and output the round trip value for the at least one tooth.

The program instructions may further include instructions to cause the processor to: identify a peak stage for the at least one tooth over the treatment plan, wherein the peak stage corresponds to a stage in the series of stages that contributes most to the round trip value; and output the peak stage for the at least one tooth. Determining the round trip value may include calculating relative movement distances of the tooth between at least some of the stages of the series of stages, and summing the relative movement distances over the treatment plan, wherein at least one of the relative movement distances has a negative value and at least one of the relative movement distances has a positive value. Determining the round trip value may include calculating a sum of movements in a positive direction greater than a planned movement, wherein the planned movement is a movement from an initial stage to a last tooth key stage of the series of stages, wherein a key stage corresponds to a stage in the series of stages where the tooth has a predetermined position. Determining the round trip value may include calculating one or more of: buccal-lingual translation, mesial-distal translation, extrusion-intrusion translation, mesial-distal rotation, mesial-distal angulation, proclination, and retroclination. Outputting the round trip value may include causing a user interface to display the round trip value.

According to an additional example, a system includes: a computing device comprising memory operationally coupled to one or more processors, wherein the memory includes instructions which can be executed by the one or more processors to cause the computing device to: receive a treatment plan for treating a patient's teeth, the treatment plan including a series of stages for sequentially moving positions of the patient's teeth from an initial arrangement to a target arrangement; determine a round trip value for at least one tooth of the patient's teeth over a course of the treatment plan, wherein the round trip value is a measure of an extent to which the at least one tooth moves in a second direction with respect to a first direction during the treatment plan, wherein the first direction is opposite the second direction; and output the round trip value for the at least one tooth.

The system may further include a display configured to display the output. The memory may further include instructions which can be executed by the one or more processors to cause the computing device to adjust the treatment plan based on the output or based on user input.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

According to one example, a method includes: determining a jaw arch from a model of a patient's upper and/or lower jaw; calculating a roundtrip value from movements relative to the jaw arch or on a tooth basis for one or more teeth of an orthodontic treatment plan comprising a plurality of different stages for moving the patient's teeth, wherein calculating the roundtrip value is performed between all of the stages, between keys of the orthodontic treatment plan, or from start to the keys of the orthodontic treatment plan, further wherein calculating the roundtrip value comprises calculating one or more of: an unplanned movement and/or a minimal total direction movement; and adjusting the orthodontic treatment plan based on the roundtrip value. Calculating the roundtrip value may include calculating buccal-lingual translations and mesial-distal translations relative to the jaw arch. Calculating the roundtrip value may include calculating a roundtrip value for extrusion-intrusion movements of the one or more teeth relative to an origin of the one or more teeth. The origin may be a crown center, root center, root apex, or tooth tip of the one or more teeth. Calculating the roundtrip value may further include calculating a roundtrip value for rotation, proclination, or retroclination movements of the one or more teeth on a tooth basis of the one or more teeth. The method may further include specifying if the roundtrip value comprises an unplanned movement, or a minimal total direction movement, or both. Calculating the roundtrip value may include generating an array with calculated roundtrip values associated with movements for the one or more teeth. The method may further include determining which stage contributes the most to the roundtrip value based on which stage is associated with the most movement. Adjusting the orthodontic treatment plan may include adjusting based on the roundtrip value and on the stage contributing the most to the roundtrip value. The method may further include forming one or more dental aligners based on the adjusted orthodontic treatment plan. Adjusting the orthodontic treatment plan may include iteratively modifying the orthodontic treatment plan to reduce the roundtrip value. The step of calculating the roundtrip value may be repeated for multiple teeth. The roundtrip value may include unplanned positive movement which is determined by summing movements in positive direction above a planned movement from an initial stage to intermediate key stages. The roundtrip value may include the minimal total direction movement which is the minimum of accumulated positive movement and negative movement of the teeth (min(Movement₊,-Movement₋) calculated between intermediate keys stages or between all stages. The stage that contributes the most to the round trip value may be a first stage that triggers the greatest roundtrip value.

According to another example, a method includes: determining a jaw arch to form a coordinate system from a model of a patient's upper and/or lower jaw; calculating a roundtrip value from movements relative to the jaw arch or on a tooth basis for one or more teeth of an orthodontic treatment plan comprising a plurality of different stages for moving the patient's teeth, wherein calculating the roundtrip value is are performed between all of the stages, between keys of the orthodontic treatment plan, or from start to the keys of the orthodontic treatment plan, further wherein calculating the roundtrip value comprises calculating one or more of: an unplanned movement and/or a minimal total direction movement; determining which stage of the plurality of different stages of the orthodontic treatment plan contributes the most to the roundtrip value; and adjusting the orthodontic treatment plan based on the roundtrip value and on the stage that contributes the most to the roundtrip value. Calculating the roundtrip value may include calculating buccal-lingual translations and mesial-distal translations relative to the jaw arch. Calculating the roundtrip value may include calculating a roundtrip value for extrusion-intrusion movements of the one or more teeth relative to an origin of the one or more teeth. The origin may be a crown center, root center, root apex, or tooth tip of the one or more teeth. Calculating the roundtrip value may further include calculating a roundtrip value for rotation, proclination, or retroclination movements of the one or more teeth on a tooth basis of the one or more teeth.

According to a further example, a method of orthodontically treating a patient's teeth includes: receiving a treatment plan for treating the patient's teeth, the treatment plan including a series of stages for sequentially moving positions of the patient's teeth from an initial arrangement to a target arrangement; determining a round trip value for at least one tooth of the patient's teeth over a course of the treatment plan, wherein the round trip value is a measure of an extent to which the at least one tooth moves in a second direction with respect to a first direction during the treatment plan, wherein the first direction is opposite the second direction; and outputting the round trip value for the at least one tooth. The method may further include: identifying a peak stage for the at least one tooth over the treatment plan, wherein the peak stage corresponds to a stage in the series of stages that contributes most to the round trip value; and outputting the peak stage for the at least one tooth. The method may further include eliminating or reducing round trip movement of the at least one tooth from the treatment plan. The method may further include calculating a new treatment plan with the eliminated or reduced round trip movement of the at least one tooth. Determining the round trip value may include calculating relative movement distances of the tooth between at least some of the stages of the series of stages, and summing the relative movement distances over the treatment plan, wherein at least one of the relative movement distances has a negative value and at least one of the relative movement distances has a positive value. Determining the round trip value may include calculating a sum of movements in a positive direction greater than a planned movement, wherein the planned movement is a movement from an initial stage to a last tooth key stage of the series of stages, wherein a key stage corresponds to a stage in the series of stages where the tooth has a predetermined position. Determining the round trip value may include calculating horizontal movements of the at least one tooth, wherein the horizontal movements include buccal-lingual translations and mesial-distal translations. Determining the round trip value may include calculating one or more of: buccal-lingual translation, mesial-distal translation, extrusion-intrusion translation, mesial-distal rotation, mesial-distal angulation, proclination, and retroclination. Outputting the round trip value may include causing a user interface to display the round trip value. The method may further include: determining a basis for evaluating movement of the at least one tooth over the treatment plan, wherein the movement of the at least one tooth is calculated with respect to an origin of the at least one tooth as a basis; and calculating extrusion-intrusion translational movements of the at least one tooth between the stages of the treatment plan using the origin as the basis. Each stage of the treatment plan may be associated with a corresponding dental appliance configured to reposition the patient's teeth according to a corresponding stage of the treatment plan.

According to another example, a non-transient, computer-readable medium contains program instructions to cause a processor to: receive a treatment plan for treating a patient's teeth, the treatment plan including a series of stages for sequentially moving positions of the patient's teeth from an initial arrangement to a target arrangement; determine a round trip value for at least one tooth of the patient's teeth over a course of the treatment plan, wherein the round trip value is a measure of an extent to which the at least one tooth moves in a second direction with respect to a first direction during the treatment plan, wherein the first direction is opposite the second direction; and output the round trip value for the at least one tooth. The program instructions may further include instructions to cause the processor to: identify a peak stage for the at least one tooth over the treatment plan, wherein the peak stage corresponds to a stage in the series of stages that contributes most to the round trip value; and output the peak stage for the at least one tooth. Determining the round trip value may include calculating relative movement distances of the tooth between at least some of the stages of the series of stages, and summing the relative movement distances over the treatment plan, wherein at least one of the relative movement distances has a negative value and at least one of the relative movement distances has a positive value. Determining the round trip value may include calculating a sum of movements in a positive direction greater than a planned movement, wherein the planned movement is a movement from an initial stage to a last tooth key stage of the series of stages, wherein a key stage corresponds to a stage in the series of stages where the tooth has a predetermined position. Determining the round trip value may include calculating one or more of: buccal-lingual translation, mesial-distal translation, extrusion-intrusion translation, mesial-distal rotation, mesial-distal angulation, proclination, and retroclination. Outputting the round trip value may include causing a user interface to display the round trip value.

According to an additional example, a system includes: a computing device comprising memory operationally coupled to one or more processors, wherein the memory includes instructions which can be executed by the one or more processors to cause the computing device to: receive a treatment plan for treating a patient's teeth, the treatment plan including a series of stages for sequentially moving positions of the patient's teeth from an initial arrangement to a target arrangement; determine a round trip value for at least one tooth of the patient's teeth over a course of the treatment plan, wherein the round trip value is a measure of an extent to which the at least one tooth moves in a second direction with respect to a first direction during the treatment plan, wherein the first direction is opposite the second direction; and output the round trip value for the at least one tooth. The system may further include a display configured to display the output. The memory may further include instructions which can be executed by the one or more processors to cause the computing device to adjust the treatment plan based on the output or based on user input.

Any of the apparatuses (e.g., systems) described herein may be configured to generate (e.g., fabricate) one or more dental appliances (e.g., aligners). The dental appliances may be generated based on three-dimensional (3D) models (e.g., 3D virtual models) of the dental appliances and/or of the patient's teeth at different stages of an orthodontic treatment plan.

In general, any of the methods described herein (or apparatuses configured to perform any of these methods) may be combined in whole or in part. For example, any of these methods may include one or more steps for ridgeline detection, e.g., for treatment planning, and/or one or more steps for intelligent staging and digital treatment planning.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows a workflow for a traditional design of a treatment plan.

FIG. 1B illustrates an example of a method for modifying and/or optimizing the treatment plan as described herein.

FIG. 2 is a diagram showing an example of a system.

FIG. 3A shows example ridgelines in relationship to various teeth.

FIG. 3B schematically illustrates processes and/or steps associated with algorithmically detecting tooth ridgelines.

FIG. 4 schematically illustrates processes and/or steps associated with determining ridgelines with a neural network.

FIG. 5 is a flowchart showing an example method 500 for training a neural network to determine dental ridgeline points.

FIG. 6 shows a 3D mesh representation of a patient's dentition.

FIG. 7 is a flowchart showing an example method for executing a neural network to predict dental ridgelines.

FIG. 8 shows a visualization of predicted probabilities associated with ridgeline points.

FIGS. 9A and 9B illustrate a conversion of ridgeline point probabilities (predictions) into ridgeline points.

FIG. 10 is a flowchart showing an example method for determining dental ridgelines.

FIG. 11 shows a block diagram of device that may be one example of a device that may be configured to perform any of the operations described herein.

FIG. 12 is a diagram illustrating one variation of a dental computing environment.

FIG. 13 is a block diagram of one example of treatment monitoring modules.

FIG. 14 is a flowchart indicating an example orthodontic treatment planning process for treating a patient's teeth.

FIG. 15 is a flowchart indicating an example process for evaluating round trips in a treatment plan.

FIG. 16 illustrates an example jaw arch indicating examples of translational movements of a tooth.

FIG. 17A is a graph indicating an example of calculating a round trip value of a tooth according to an unplanned movement.

FIG. 17B is a graph indicating an example of calculating a round trip value of a tooth according to a minimal total direction movement.

FIG. 18 is a block diagram of an example system for calculating round trip values for a treatment plan.

FIG. 19 illustrates an example user interface for digital treatment planning.

### DETAILED DESCRIPTION

The methods and apparatuses described herein may determine one or more dental ridgelines. These methods and apparatuses (e.g., systems, including software, hardware and/or firmware) may include the use of a trained neural network and/or training and execution of a neural network that identifies one or more dental ridgelines. In some examples, the neural network can be trained to identify location points that lie on a dental ridgeline. The dental ridgeline can pass through the identified location points. The dental ridgelines may be used to determine a tooth's position in a jaw bone. Accurate tooth position may be used to determine a dental treatment plan that can, in turn, be used to predict the final position of teeth.

In contrast with current algorithmic approaches, the methods and apparatuses described herein are not dependent on the use of additional data that may influence the position of detected points, for example, the position of one or more teeth axes, because the methods described herein use primarily the raw geometry of the dentition. These methods and apparatuses may be more robust than previously described techniques for determining ridgelines in teeth, as they may more accurately identify required points and positions from teeth in cases of unusual teeth geometry, and in conditions in which there may be problems with scan and segmentation quality. As a result, the methods and apparatuses described herein may result in better proposed final positions for one or more orthodontic treatments, including treatments that are more likely to be acceptable to the dental professional. This is possible because ridgelines are one feature that is used to generate treatment plans, and the methods an apparatuses described herein may generate more accurate and robust estimates of ridgelines, even in difficult cases. Surprisingly, these methods may use less input data than more traditional methods, in some cases using only teeth shape without requiring information about the axes.

In general, these methods and apparatuses may apply a trained neural network to a scan and/or 3D model of the patient's dentition in order to generate ridgeline data that may be used for treatment planning. As will be described in greater detail herein, the neural networks may be trained with labeled images of teeth that include ridgelines. The neural network may more accurately determine dental ridgelines particularly for teeth that have irregular shapes. In some variations, the neural network may be used in combination with a conventional algorithmic approach to determine dental ridgelines. For example, if a tooth is regularly shaped, a conventional algorithm can be used to determine ridgelines. If, on the other hand, a tooth is irregularly shaped (e.g., chipped, broken, partially occluded), then a neural network can be used to determine ridgelines. Thus, in some cases these methods and apparatuses may provide logic to determine when (and how) to apply the trained neural network to best determine ridgelines for one or more teeth, based on the tooth morphology.

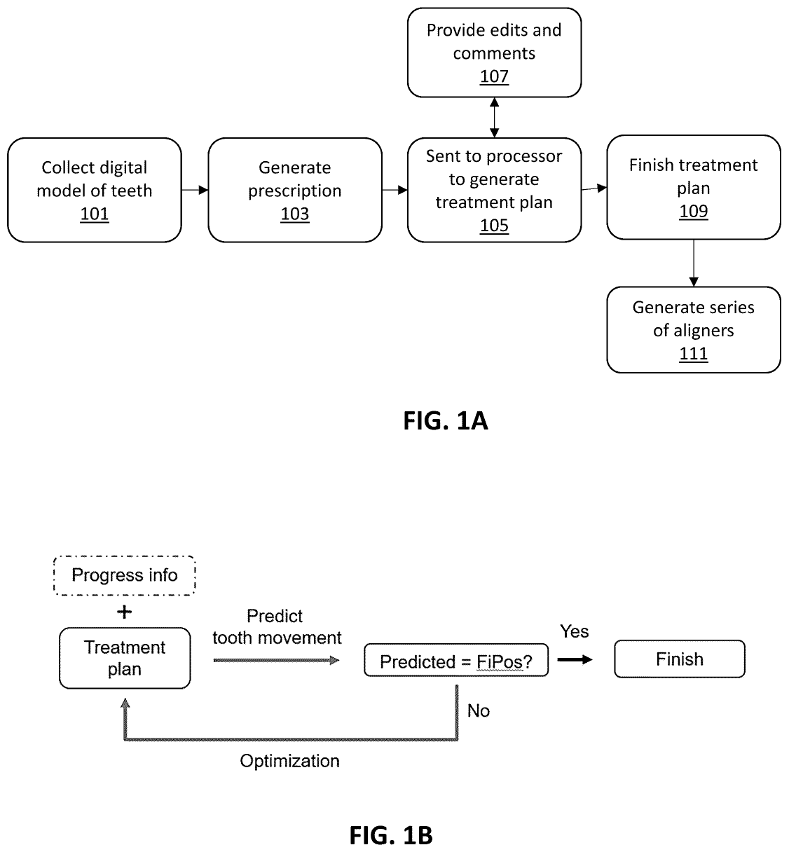

FIG. 1A shows a workflow for a traditional design of a treatment plan (sometimes referred to as a digital treatment plan). In this example, the patient's teeth are digitally modeled, e.g., by scanning the teeth direction (e.g., using an intraoral scanner) and/or by scanning an impression of the teeth, etc., 101. The user may then generate a prescription, e.g., indicating the treatment targets and/or goals, as well as instructions on the treatment steps (e.g., the use of attachments, interproximal reductions, number of stages, etc.), 103 and this information may be transmitted to a processor that may generate a treatment plan 105. For example, the treatment plan may be generated by one or more modules (e.g., “FiPos”) that generates the final position. This target final position (“target position”) may be reviewed and iterated by the user and manually, automatically or semi-automatically modified 107. For example, the user may provide edits on the final positions and/or treatment steps. Once this process is completed, the final treatment plan may be generated 109 and, after final approval/review, a series of treatment aligners may be generated 111.

FIG. 1B illustrates an example of a method for modifying and/or optimizing the treatment plan as described herein. In this example, an initial treatment plan (e.g., from or at step 105 or 109 in FIG. 1A) may be optimized as described herein, for example, by using the initial patient tooth positions, the treatment plan and target tooth positions along with a regression model. The regression model may be used with the patient characteristic (e.g., demographical info such as age, gender etc., and/or clinical and/or cosmetic concerns) to select the regression model. The regression model may be referred to as the trained prediction mode, and may predict the tooth movement at the end of the treatment.

The methods or apparatus may also include checking whether the predicted tooth movements match with the desired tooth movement (i.e. FiPos) that users set. If there is a mismatch between the predicted tooth movement (e.g., predicted tooth position) and the desired tooth movement (e.g., target tooth position), the method or apparatus may modify the treatment plan with the optimization module, and may predict the tooth movement again based on the updated treatment plan. These optimization iterations may continue until the prediction of the tooth movements match with the prescribed target tooth movements (or target tooth positions, as generated by “FiPos”). Once the optimization iterations are completed, the method or apparatus may generate a series of aligners based on the optimized treatment plan.

For existing patients, the method or apparatus may leverage the scans from the patient's previous treatment (e.g., progress info) to account for the individual differences, for example, in wearing habits, bone physiology etc., into the regression model, and may further improve the tooth movement prediction. This is illustrated in FIG. 1B (“Progress info”). For example, to design the treatment plan for an existing patient, the method or apparatus may first extract the progress info from the patient (e.g., planned/achieved tooth movement from previous treatments), and may then utilize the progress info along with the current treatment plan and the one or more patient characteristic(s) (e.g., demographical info such as age, gender etc., and/or clinical concerns, and/or cosmetic concerns) as inputs to the regression model to predict the tooth movement at the end of the treatment. The method or apparatus may then follow the same optimization process described above, generating a predicted tooth movement (or equivalently in this context, tooth position), and determining a difference indicator between the two for each tooth of the dental arch in the multiple translational/rotational movements (e.g., buccal/lingual, mesial/distal, and intrusion/extrusion movements).

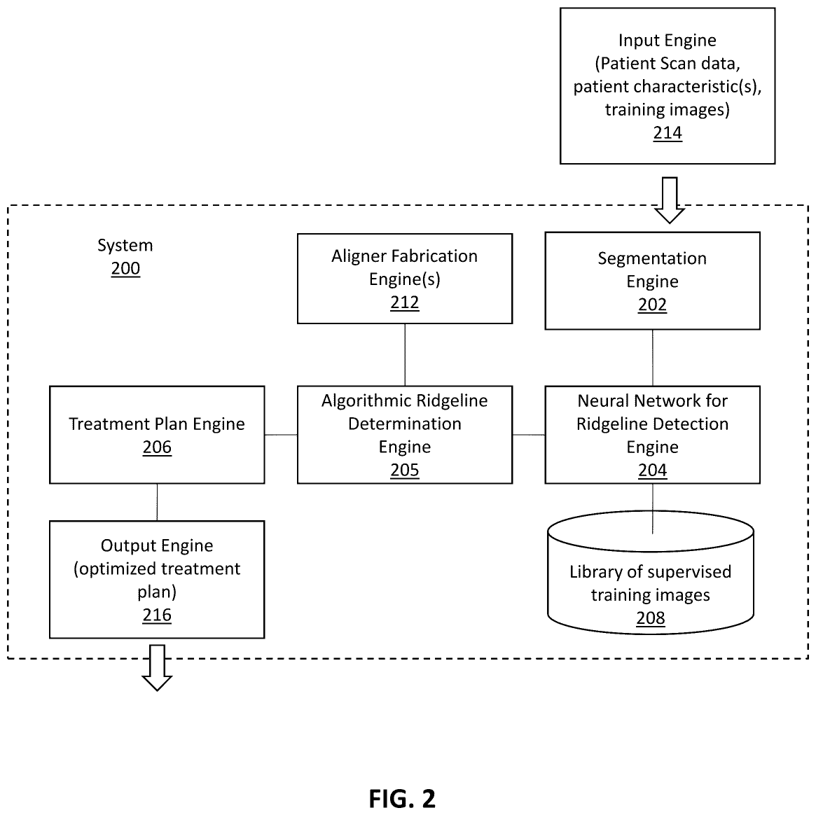

FIG. 2 is a diagram showing an example of a system 200; the system 200 may be incorporated into a portion of another system (e.g., a general treatment planning system, as described below) and may therefore also be referred to as a sub-system. Alternatively the methods and apparatuses for performing them described herein may be included as part of a different system. In any of the methods and apparatuses described herein, the system 200 may be invoked by a user control, such as a tab, button, etc., as part of treatment planning system, or may be separately invoked.

In FIG. 2, the system 200 may include a plurality of engines and datastores. A computer system can be implemented as an engine, as part of an engine or through multiple engines. As used herein, an engine includes one or more processors, or a portion thereof. A portion of one or more processors can include some portion of hardware, less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, may be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently, within a given context. Data structures are generally based on the ability of a computer to fetch and store data at any place in its memory, specified by an address, a bit string that can be itself stored in memory and manipulated by a program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores described herein can be cloud-based datastores. A cloud-based datastore is a datastore that is compatible with cloud-based computing systems and engines.

The system 200 may include or be part of a computer-readable medium, and may include an input engine 214 (e.g., providing and/or allowing access to the patient's scan data, and/or patient characteristic(s). The scan data may include three-dimensional (3D) scan data provided by an intraoral scanner, or the like. In some embodiments, the input engine 214 may receive training images, including supervised training images. As will be described herein, the training images may be used to train one or more neural networks.

The system 200 may include a segmentation engine 202 that may segment 3D models into different objects, sections, parts, or the like. Segmentation may be performed in any feasible manner. One of example of segmentation is described in U.S. Pat. No. 11,020,206, which is incorporated by reference in its entirety. In some variations, the segmentation engine 202 may also process (e.g., convert, transform) 3D scan data into a 3D model. For example, the 3D scan data can include point cloud data of the patient's dentition. Each point in the point cloud can represent a point in 3D space of the patient's dentition, as captured, for example, by an intraoral scanner. Thus, the segmentation engine 202 can receive 3D scan data from the input engine 214, generate 3D models of the patient's dentition and then segment the 3D models into separate objects, sections, parts, or the like. In some variations, the segmentation engine 202 can segment the 3D models into teeth and gingiva. In some examples the system 200 may include a memory, register or datastore storing all or some of the patient's initial tooth position, and/or patient characteristic(s). Patient characteristics may include patient demographical info such as age, gender and the like.

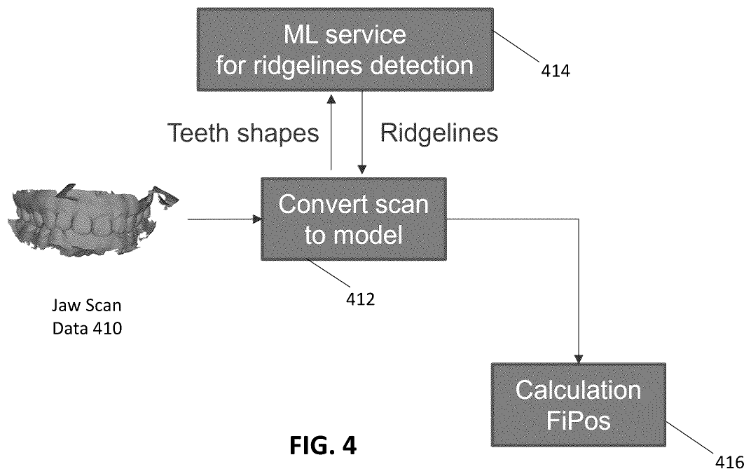

The system 200 may also include a neural network for ridgeline detection engine 204. All teeth can include one or more ridgelines that may be used to determine a tooth's position and/or orientation with respect to a jaw bone and/or other teeth. The neural network for ridgeline detection engine 204 may be trained to detect and identify dental ridgelines on a patient's teeth. The neural network for ridgeline detection engine 204 may more easily detect and identify dental ridgelines for irregular (broken, chipped, partially occluded, etc.) teeth in comparison to other approaches. The tooth's position and/or orientation may be used, at least in part, to determine a patient's treatment plan. In some variations, a patient's treatment plan may include determining the final position of the patient's teeth (e.g., the position of the patient's teeth at completion of dental treatment). In some variations, the system 200 may store a library of supervised training images 208. These images may be used to train the neural network for ridgeline detection engine 204. The neural network for ridgeline detection engine 204 is described in more detail below in conjunction with FIGS. 4-8.

The system 200 may include an algorithmic ridgeline determination engine 205. The algorithmic ridgeline determination engine 205 can detect and determine the ridgelines of one or more teeth without using neural networks. For example, 3D scan data can be projected onto a two-dimensional plane to form a height map, and points from the height map can be used to determine dental ridgelines. Generally, the algorithmic ridgeline determination engine 205 works best with “regular” teeth. That is, teeth that are not broken, chipped, partially occluded, etc. As described above, determined ridgelines may be used to determine tooth position and orientation which may be used in determining a patient's treatment plan. One of example of algorithmic ridgeline detection is described in U.S. Pat. No. 7,774,339, which is incorporated by reference in its entirety.

The system 200 may include a treatment plan engine 206. The treatment plan engine 206 may process patient scan data, tooth ridgeline data from the neural network for ridgeline detection engine 204 and/or the algorithmic ridgeline determination engine 205, patient characteristics, clinician input and the like to determine a patient's treatment plan. In some variations, the patient's treatment plan may be used to move a patient's teeth using a series of aligners that may be worn by the patient. Each aligner may incrementally move a tooth's position by applying pressure to one or more teeth. In some variations, the treatment plan engine 206 can generate aligner data to manufacture associated dental aligners in accordance with the treatment plan. Thus, any of these apparatuses may include an output engine 216 for outputting the treatment plan.

The aligner fabrication engine(s) 212 may implement one or more automated agents configured to fabricate an aligner. Examples of an aligner are described in detail in U.S. Pat. No. 5,975,893, and in published International Patent Application No. WO 98/58596, which is herein incorporated by reference for all purposes. Systems of dental appliances employing technology described in U.S. Pat. No. 5,975,893 are commercially available from Align Technology, Inc., Santa Clara, Calif., under the tradename, Invisalign System. Throughout the description herein, the use of the terms “orthodontic aligner”, “aligner”, or “dental aligner” is synonymous with the use of the terms “appliance” and “dental appliance” in terms of dental applications. For purposes of clarity, embodiments are hereinafter described within the context of the use and application of appliances, and more specifically “dental appliances.” The aligner fabrication engine(s) 212 may be part of 3D printing systems, thermoforming systems, or some combination thereof. In some variations, the aligner fabrication engine(s) 212 can process the aligner data provided by the treatment plan engine 206 to generate one or more dental aligners.

One or more of the engines of the system 200 may be coupled to one another (e.g., through the example couplings shown in FIG. 2) or to modules/engines not explicitly shown in FIG. 2. The computer-readable medium may include any computer-readable medium, including without limitation a bus, a wired network, a wireless network, or some combination thereof.

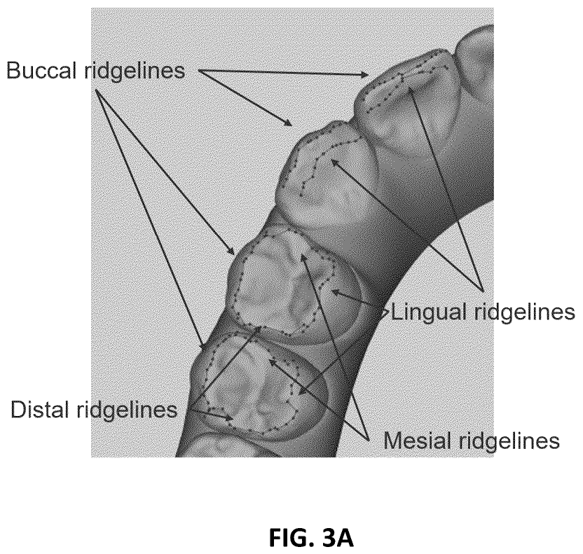

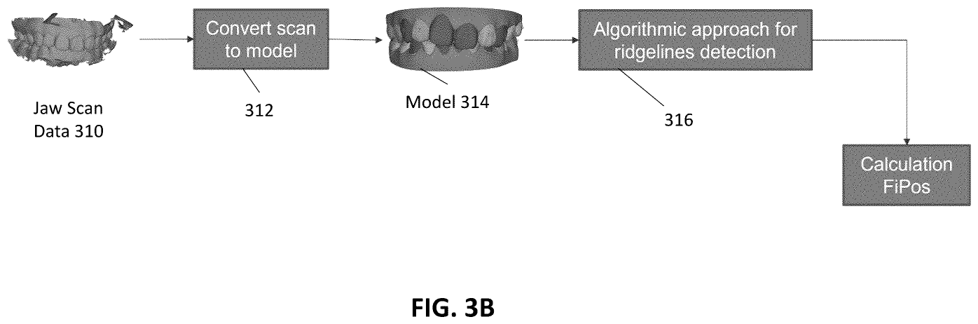

FIG. 3A shows example ridgelines in relationship to various teeth. As shown, all teeth can have at least one kind of ridgeline, however all teeth may not have the same number or types of ridgelines. In some cases, determination of one or more ridgelines may be made more difficult, particularly if a tooth is irregularly shaped, chipped or broken, or occluded by gingiva or other anatomies.

FIG. 3B schematically illustrates processes and/or steps associated with algorithmically detecting tooth ridgelines and determining a final tooth position from the tooth ridgelines. In some examples, a system (such as the system 200 of FIG. 2) can receive jaw scan data 310. The jaw scan data can include 3D scan data from an intraoral scanner, 3D scan data from other devices, 2D scan data, or any other feasible scan data of the patient's detention. In some variations, the jaw scan data can include point cloud data of the patient's dentition.

Next, jaw scan data is converted (at block 312) to a 3D model shown at block 314. In some cases, conversion to the 3D model may include segmentation of the 3D model into individual teeth, bones, gingiva, or the like.

Next, in block 316 ridgelines associated with one or more teeth may be determined. The ridgelines may be algorithmically determined as described with respect to the algorithmic ridgeline determination engine 205 of FIG. 2. The ridgelines may be used to determine tooth positions that, in turn, may be used to determine a final position (FiPos) at block 316. In some examples, a related treatment plan can also be determined at block 316.

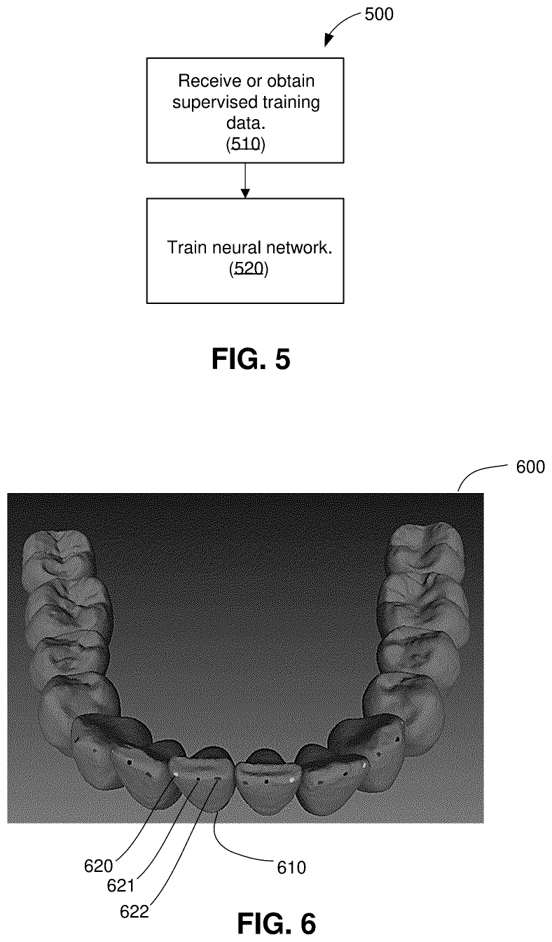

FIG. 4 schematically illustrates processes and/or steps associated with determining dental ridgelines with a neural network and determining a final tooth position from the determined ridgelines. In general, the final position of teeth may be determined based on a treatment plan. As described below in conjunction with FIGS. 5-10, tooth ridgelines may be determined from jaw scan data, such as 3D jaw scan data. The jaw scan data can be converted into a model (e.g., a 3D model) that can include tooth shapes for one or more teeth. In some examples, a trained neural network (also referred to as a trained machine learning agent) may be executed to determine ridgelines that are included within one or more teeth of the 3D model.

As described above, some ridgelines may be more difficult to detect or locate, particularly when the ridgelines are associated with irregularly shaped teeth, chipped or broken teeth, occluded teeth, or the like. A neural network, trained with models that include irregularly shaped, chipped, broken, and/or occluded teeth, may more accurately locate and/or identify dental ridgelines.

Jaw scan data 410 is converted to a 3D model at block 412. The 3D model can include a point cloud data representation of the patient's dentition. Segmented teeth shapes from the 3D model are provided to a neural network for determining ridgelines at block 414. The ridgelines are returned to block 412 and are used to more accurately determine tooth position. The tooth position is then used in block 416 to determine a final position.

FIG. 5 is a flowchart showing an example method 500 for training a neural network to determine dental ridgeline points (sometimes referred to as ridgeline point locations). Some examples may perform the operations described herein with additional operations, fewer operations, operations in a different order, operations in parallel, and some operations differently. Dental ridgeline points are points which lie on, or are otherwise included with, ridgelines on a patient's tooth. Thus, various ridgeline points may be used to determine the location of the related ridgelines. Any of the neural networks described herein may be used to locate one or more ridgeline points. The method 500 is described below with respect to the system 200 of FIG. 2, however, the method 500 may be performed by any other suitable system or device.

In block 510, the system 200 receives supervised training data. Supervised training data can include images that have been manually labeled by skilled personnel. The labeled images include any and all points (locations) or other characteristics that the neural network will be trained to recognize. In some variations, the supervised training data can be labeled to train a neural network to operate on upper and lower jaws separately. The neural network can include a first channel (or classifier) that is trained to identify (e.g., determine the probabilities associated with regions of the patient's 3D model) tooth type (tooth number) and jaw bone. Since any one jaw can include up to 16 teeth, the neural network can be trained to identify 16 teeth as well as the jaw bone. This is sometimes referred to as a neural network channel trained to classify 17 different classes.

The neural network can also include a second channel (or classifier) that is trained to identify dental ridgeline points. For example, the second channel can identify dental ridgeline points that include edge mesial, edge distal, and edge middle points. The second channel can also identify non-ridgeline points (e.g., points that do not lie on the dental ridgeline). Thus, the second channel can be trained to classify four different classes (three ridgeline points and points not on a ridgeline). As mentioned above, any of the methods and apparatuses described herein may use multiple (e.g., two, three, etc.) channels, such as classifier of tooth type (tooth number) and/or classifier of ridgeline points. In any of these methods and apparatuses, the method may include the use of a single channel that can segment jaw scans and create jaw model with known tooth type (e.g., which may provide a labeled segmented 3D models, with known tooth type) with ridgeline points.

In general, the process of making predictions may be referred to herein as the classification of ridgelines points. This may also be a segmentation problem. For example, in some examples the method may include segmenting triangles (shapes) before classifying by tooth type in the 3D model and/or after, e.g., by postprocessing transformation of the segmentation by ridgelines classes to points location as described above. These processes may also or alternatively be done on 3D point cloud and/or on 2D images, as it was mentioned herein. Any of these method may attempt to segment an area by some class.

In some variations, the supervised training data is “balanced.” That is, the supervised training data should include cases with regular anatomy and unusual anatomy in approximately equal proportions. In some cases, some of the training data can be “spoiled” or manipulated to transform regular tooth anatomies into unusual tooth anatomies. In some examples, the supervised training data can include between 30,000 and 50,000 different cases.

In block 520, the system 200 trains the neural network with the supervised training data received in block 510. As described above, the system 200 can train the neural network (machine learning agent) to respond with two channels. A first channel can identify teeth and jaw bones and a second channel can identify ridgeline points. In some embodiments, the neural network is trained to generate two point cloud files: one file for the upper jaw and one file for the lower jaw. The neural network can be trained to respond with the first and second channels to identify the classes described herein within the point cloud files.

The training of a neural network to identify or locate dental ridgelines may be associated with various aspects of a dental computing environment that is used to determine dental treatment plans and, in some cases, generate one or more dental appliances used to implement a dental treatment plan. By way of example and not limitation, the training of a neural network may be associated with treatment planning or a treatment planning system. For example, a treatment planning system may include one or more modules configured to receive or obtain supervised training data and determine or train a neural network to identify or locate dental ridgelines based on the supervised training data.

In some embodiments, the neural network can be trained to identify the classes from triangles (triangle shapes) that are included in the patient's 3D model. Thus, patient's 3D model can be transformed from a point cloud representation into a 3D mesh. In some embodiments, every point in a point cloud of the patient's 3D model may be assigned to a vertex of a triangle. If multiple triangles rely on a common vertex, then the common vertex may be associated with a ridgeline point. In some variations, a ridgeline point may be associated with a vertex that is common to approximately between six and eight triangles. In some other variations, a ridgeline point may be associated with any predetermined number of triangles.

FIG. 6 shows a 3D mesh representation of a patient's dentition 600. As described in FIG. 5, a point cloud representation of the patient's dentition 600 may be transformed into the 3D mesh representation. The patient's dentition 600 also includes the location of ridgeline points for some teeth. For example, tooth 610 can include edge mesial 620, and edge middle 621, and edge distal 622 ridgeline points.

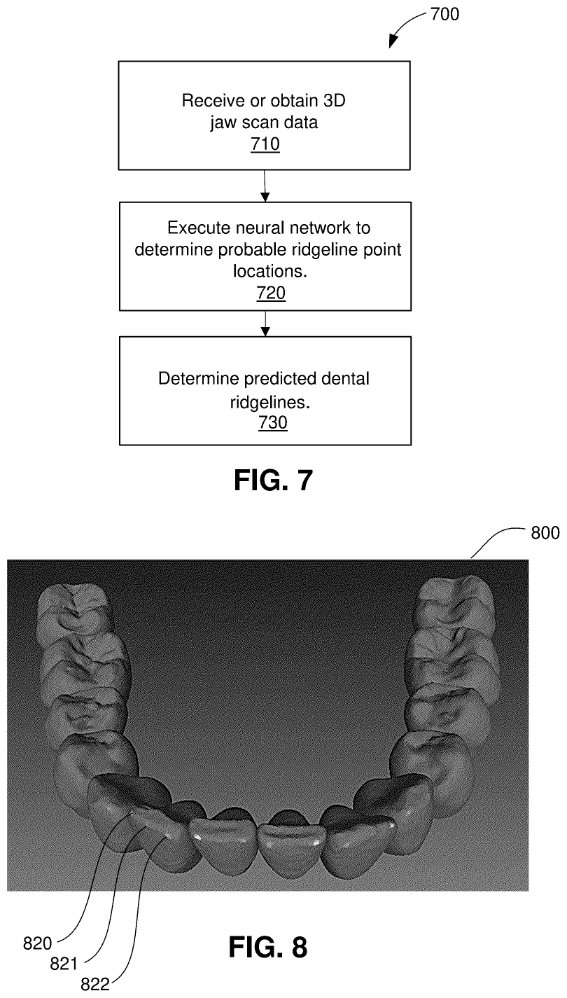

FIG. 7 is a flowchart showing an example method 700 for executing a neural network to predict dental ridgelines. The method 700 is described below with respect to the system 200 of FIG. 2, however, the method 700 may be performed by any other suitable system or device.

The method 700 begins in block 710 as the system 200 receives or obtains a patient's 3D jaw scan data. In some variations, the 3D jaw scan data can be point cloud data from an intraoral scanner. In some other variations, the 3D jaw scan data can be provided by any other feasible device or scanner.

Next, in block 720 the system 200 can execute a neural network to determine probable ridgeline point locations. For example, the system 200 can transform the 3D jaw scan data into a 3D mesh of triangles. The system 200 can then execute a neural network to determine possible ridgeline points as well as the probabilities that any of the possible ridgeline points are actual ridgeline points. The neural network may be trained as described with respect to FIG. 5.

In some cases, the neural network can determine, for each triangle vertex, the probability that the vertex is associated with a dental ridgeline.

Next, in block 730, the system 200 can determine predicted dental ridgeline based on the possible ridgeline points identified in block 720. As the probabilities for all vertices are examined, those with the highest probability may be identified. Those identified vertices may then be examined to determine if they are associated with a ridgeline point.

The method 700 may advantageously locate dental ridgelines with respect to teeth that may otherwise not respond to an algorithmic approach. The execution of a neural network to identify or locate dental ridgelines may be associated with various aspects of a dental computing environment. For example, execution of the neural network may be associated with a treatment planning system, an intraoral scanning system, or any other feasible system. For example, an intraoral scanning system may include one or more modules to determine simulation outcomes. In some cases simulation outcomes may be based on an initial tooth location determined through dental ridgelines. In another example a treatment planning system can use scan information (3D scanning information) to attain 3D scan information and determine one or more dental ridgelines therefrom. In either case, the dental ridgelines may be used to determine a final position of teeth in accordance with a dental treatment plan.

FIG. 8 shows a visualization of predicted probabilities associated with ridgeline points 800. In some embodiments, a neural network may identify a plurality of points as possible edge distal, edge middle, and edge mesial ridgeline points. To illustrate this family of points, FIG. 8 shows some ridgeline points with varying intensity. The greater the intensity, the greater the probability that any one point is a ridgeline point. For example, in FIG. 8 the tooth shows edge mesial 820, edge middle 821, and edge distal 822 ridgeline points with different intensities.

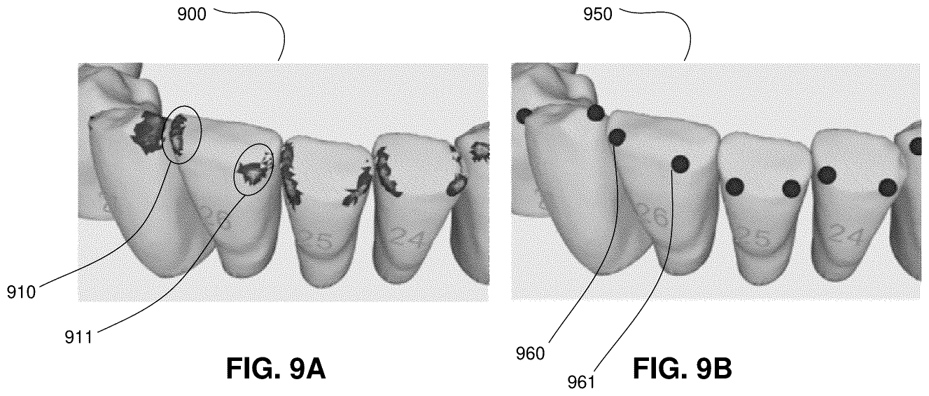

FIGS. 9A and 9B illustrate a conversion of ridgeline point probabilities (predictions) into ridgeline points. FIG. 9A shows teeth included on a portion of a patient's lower jaw 900. The probability associated with particular vertices (triangle vertices) is shown by a varying intensity. The varying intensities can be a “heat map” showing a probable ridgeline point (an intense color shows more vertices that may be possible ridgeline points. For example, region 910 shows a distribution of probabilities of a first ridgeline point and region 911 shows a distribution of probabilities of a second ridgeline point. Only two possible ridgeline points are shown here for simplicity. In other embodiments, any tooth can include any number of possible ridgeline points.

FIG. 9B shows example ridgeline points the teeth of a patient's lower jaw 950. Each region in FIG. 9A that included a distribution of probabilities has been converted into a ridgeline point. In some embodiments, the regions of distributed probabilities are examined, and the highest probability belonging to each class of ridgeline point (edge mesial, edge middle, or edge distal) is identified as a ridgeline point. In some examples, a ridgeline point is based on a center mass of the probabilities within a region. For example, the probabilities of region 910 of FIG. 9A is analyzed to identify ridgeline point 960. Similarly, the region 911 of FIG. 9A is analyzed to identify ridgeline point 961.

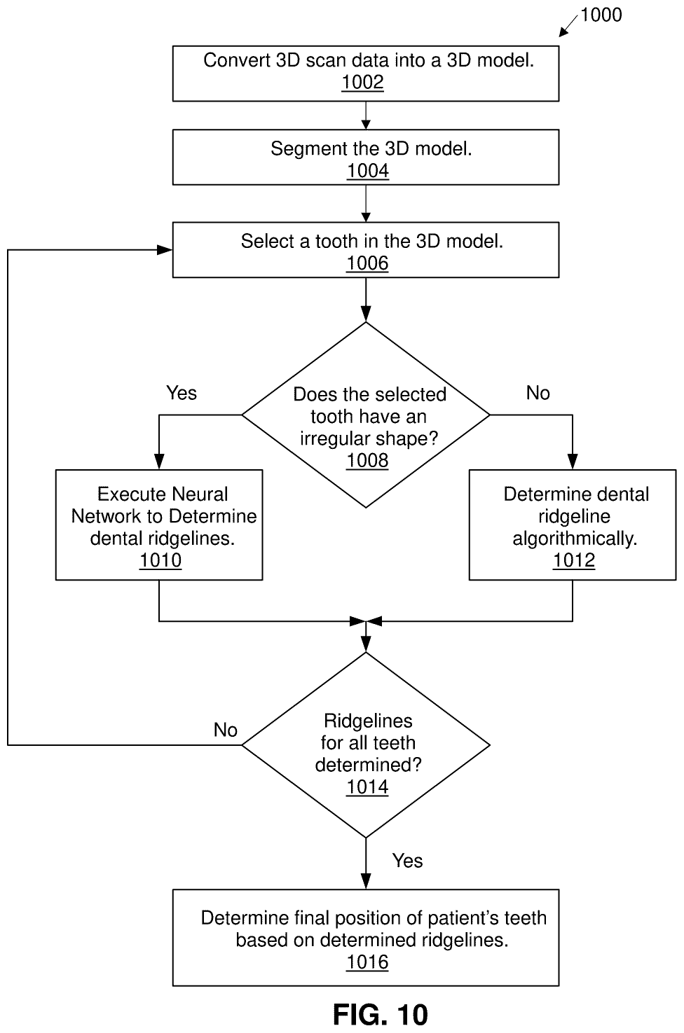

FIG. 10 is a flowchart showing an example method 1000 for determining dental ridgelines. The method 1000 is described below with respect to the system 200 of FIG. 2, however, the method 1000 may be performed by any other suitable system or device.

The method 1000 begins in block 1002 as the system 200 converts 3D scan data into a 3D model. In some variations, the 3D scan data can be a point cloud of 3D jaw scan data. The system 200 can transform the point cloud data into a 3D model that includes a 3D mesh of triangles.

Next, in block 1004, the system 200 segments the 3D model. In some examples, the system 200 can segment the 3D model into individual teeth and related jaw bones. In some cases, the system 200 can execute a neural network, as described above, to segment the 3D model into included teeth, bones, and gingiva.

Next, in block 1006 the system 200 selects a tooth in the 3D model. The selected tooth can be any tooth in the 3D model. In block 1008, the system 200 determines if the selected tooth has an irregular shape.

If the selected tooth has an irregular shape, then in block 1010 the system 200 executes a neural network to determine dental ridgelines. In some examples, the system 200 can perform the method 700 of FIG. 7 to execute a neural network to determine dental ridgelines.

The method 700 proceeds to block 1014 where the system 200 determines if ridgelines for all teeth have been determined. If ridgelines for all the teeth have not been determined, then the method 700 returns to block 1006 where another tooth is selected. If, on the other hand, the ridgelines for all the teeth have been determined, then in block 1016 the system 200 determines a final position of the patient's teeth. As described with respect to FIG. 3B and FIG. 4, the system 200 can determine tooth positions from dental ridgeline information, and furthermore, can determine final tooth positions as well as a dental treatment plan.

Returning to block 1008, if a selected tooth does not have an irregular shape, then the method 700 proceeds to block 1012 where the system 200 determines dental ridgelines for the selected teeth algorithmically. In some examples, the system 200 can determine dental ridgelines as described above with respect to FIG. 3B. The method then proceeds to block 1014.

The method 1000 advantageously adapts to different methods for determining dental ridgelines to different teeth of the patient. Irregular teeth are automatically processed by a neural network, which is well suited to identify ridgelines of irregular teeth. Regular teeth are automatically processed by algorithmically based routines which may provide good results for regular teeth.

The method 1000 of FIG. 10 (in a similar manner as described above with respect to FIG. 7) may be associated with various aspects of a dental computing environment. For example, the method 1000 may be associated with a treatment planning system, an intraoral scanning system, or any other feasible system.

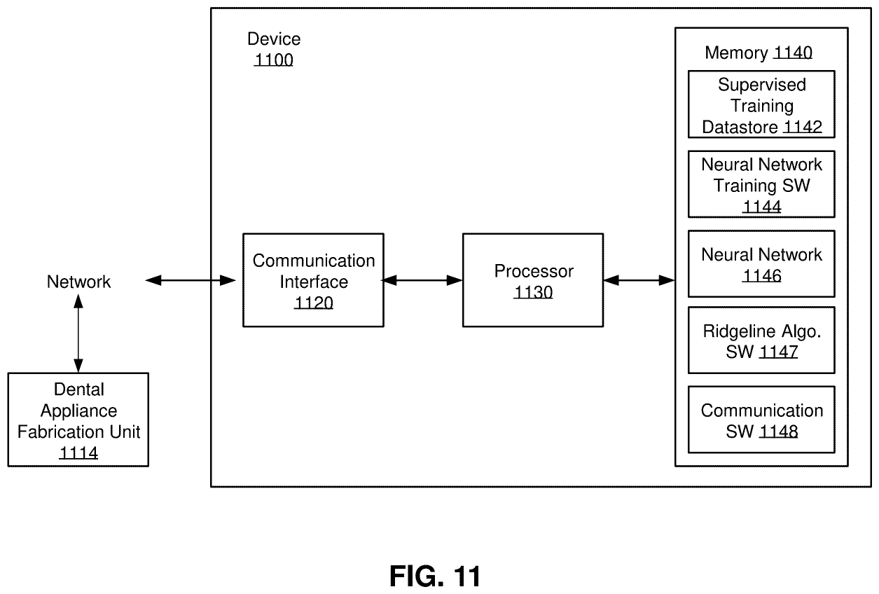

FIG. 11 shows a block diagram of device 1100 that may be one example of a device that may be configured to perform any of the operations described herein. The device 1100 may include a communication interface 1120, a processor 1130, and a memory 1140. The device 1100 may be local (near) the user (clinician) that wants to determine dental appliance (e.g., dental aligner) data. In some variations, the device 1100 may be remote (separate) from the user. For example, the device 1100 may be implemented as a server or may be distributed on two or more servers or may be cloud (internet) based.

The communication interface 1120, which is coupled to the processor 1130, which may be coupled to a network and to the processor 1130, may transmit signals to and receive signals from other wired or wireless devices, including remote (e.g., cloud-based) storage devices, cameras, processors, compute nodes, processing nodes, computers, mobile devices (e.g., cellular phones, tablet computers and the like) and/or displays. For example, the communication interface 1120 may include wired (e.g., serial, ethernet, or the like) and/or wireless (Bluetooth, Wi-Fi, cellular, or the like) transceivers that may communicate with any other feasible device through any feasible network. In some examples, the communication interface 1120 may receive a patient's scan data, training images, or the like

The communication interface 1120 may be coupled directly or indirectly to a dental appliance fabrication unit 1114. The dental appliance fabrication unit 1114 may receive dental appliance data generated by the device 1100 and, in turn, generate dental appliances. In some cases, the dental appliance data from the device 1100 may be used to generate dental aligners, including clear dental aligners.

The processor 1130, which is also coupled to the memory 1140, may be any one or more suitable processors capable of executing scripts or instructions of one or more software programs stored in the device 1100 (such as within memory 1140).

The memory 1140 may include a supervised training datastore 1142. The supervised training datastore 1142 may include a quantity of labeled images that may be used to train one or more neural networks. In some embodiments, supervised training datastore 1142 can include images of a patient's dentition that have been labeled with teeth and jawbones. In some other embodiments, the supervised training datastore can include images of teeth labeled with dental ridgeline points include mesial edge, middle edge, and distal edge ridgeline points.

The memory 1140 may also include a non-transitory computer-readable storage medium (e.g., one or more nonvolatile memory elements, such as EPROM, EEPROM, Flash memory, a hard drive, etc.) that may store the following software modules: a neural network training software (SW) module 1144 to train a neural network 1146; a ridgeline algorithm SW module 1147, and a communication SW module 1148.

Each software module, module, or engine includes program instructions that, when executed by the processor 1130, may cause the device 1100 to perform the corresponding function(s). Thus, the non-transitory computer-readable storage medium of memory 1140 may include instructions for performing all or a portion of the operations described herein.

The processor 1130 may execute the neural network training SW module 1144 to train the neural network 1146. In some examples, execution of the neural network training SW module 1144 may cause the processor 1130 to train the neural network 1146 using data in the supervised training datastore 1142. Execution of the neural network training SW module 1144 can train the neural network 1146 in accordance with the method 500 of FIG. 5.

The processor 1130 may execute the ridgeline algorithm SW module 1147 to determine and/or locate dental ridgeline algorithmically. In some variations, execution of the ridgeline algorithm SW module 1147 may locate dental ridgeline in accordance with the steps described in FIG. 3B.

The processor 1130 may execute the communication SW module 1148 to communicate with any other feasible devices. For example, execution of the communication SW module 1148 may enable the device 1100 to communicate via cellular networks conforming to any of the LTE standards promulgated by the 3rd Generation Partnership Project (3GPP) working group, Wi-Fi networks conforming to any of the IEEE 802.11 standards, Bluetooth protocols put forth by the Bluetooth Special Interest Group (SIG), Ethernet protocols, or the like. In some embodiments, execution of the communication SW module 1148 may enable the device 1100 to communicate, directly or indirectly, with the dental appliance fabrication unit 1114. In some other embodiments, execution of the communication SW module 1148 may implement encryption and/or decryption procedures.

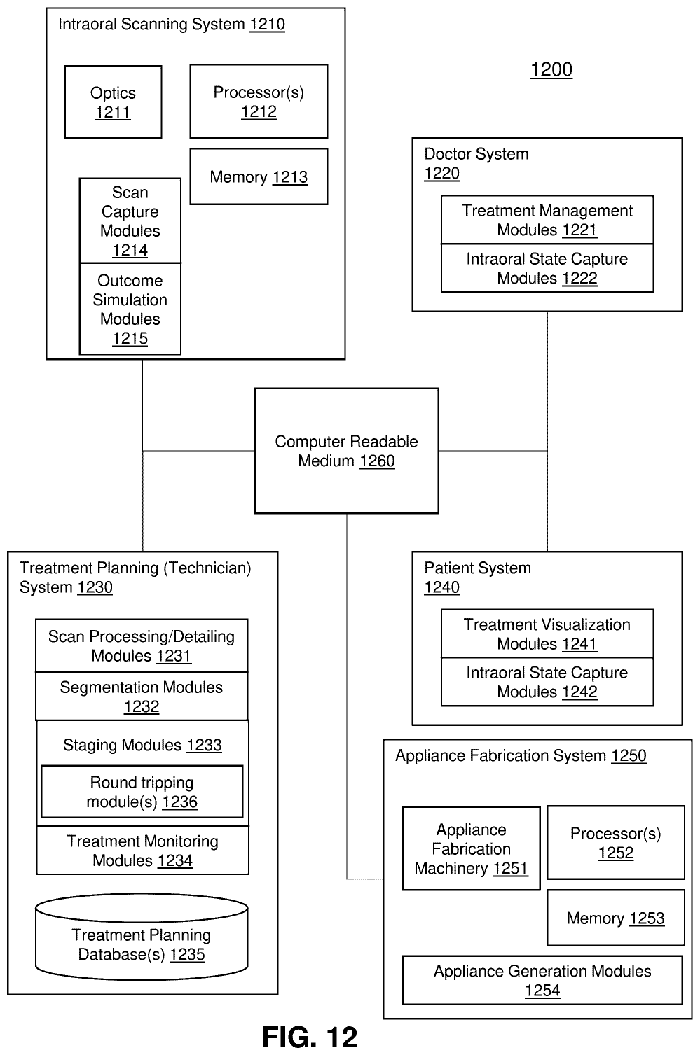

In general, the methods and apparatuses described herein for determining one or more ridgelines may be used at one or more parts of a dental computing environment, including as part of an intraoral scanning system, a doctor system, a treatment planning system, and/or a fabrication system. In particular, these methods and apparatuses may be used as part of a treatment planning system, for example, to determine an accurate (in some cases initial) location of a patient's teeth. The initial location may be used to determine a final location for one or more of the patient's teeth based on a treatment plan. For example, FIG. 12 is a diagram illustrating one variation of a dental computing environment 1200 that may generate one or more orthodontic treatment plans specific to a patient, and fabricate dental appliances that may accomplish the treatment plan to treat a patient, under the direction of a dental professional. The example dental computing environment 1200 shown in FIG. 12 includes an intraoral scanning system 1210, a doctor system 1220, a treatment planning system 1230 (e.g., technician system), a patient system 1240,, an appliance fabrication system 1250, and computer-readable medium 1260. In some variations the dental computing environment (dental computing system) 1200 may include just one or a subset of these systems (which may also be referred to as sub-systems of the overall system 1200). Further, one or more of these systems may be combined or integrated with one or more of the other systems (sub-systems), such as, e.g., the treatment planning system 1230 and the doctor system 1220 may be part of a remote server accessible by a doctor interface. The computer readable medium 1260 may divided between all or some of the systems (subsystems); for example, the treatment planning system 1230 and appliance fabrication system 1250 may be part of the same sub-system and may be on a computer readable medium 1260. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, the intraoral scanning system 1210 can include lens(es) 1211, processor(s) 1212, a memory 1213, scan capture modules 1214, and outcome simulation modules 1215. In general, the intraoral scanning system 1210 can capture one or more images of a patient's dentition. Use of the intraoral scanning system 1210 may be in a clinical setting (doctor's office or the like) or in a patient-selected setting (the patient's home, for example). In some cases, operations of the intraoral scanning system 1210 may be performed by an intraoral scanner, dental camera, cell phone or any other feasible device.

The lens(es) 1211 include one or more lenses and optical sensors to capture reflected light, particularly from a patient's dentition. The scan capture modules 1214 can include instructions (such as non-transitory computer-readable instructions) that may be stored in the memory 1213 and executed by the processor(s) 1212 to control the capture of any number of images of the patient's dentition.

As mentioned, in some examples the methods and apparatuses described herein for generating a 3D model including one or more teeth may be part of, or accessible by, the intraoral scanning system 1210, computer readable medium 1260 and/or treatment planning system 1230.

For example, the outcome simulation modules 1215, which may be part of the intraoral scanning system 1210, can include instructions that simulate final tooth positions based on a treatment plan. In some cases, the outcome simulation modules 1215 can include instructions that simulate tooth positions using an initial tooth position based on determined dental ridgelines. In some examples, the outcome simulation modules 1215 may include a trained neural network that can determine dental ridgelines from 3D models provided by the scan capture modules 1214.

Any of the component systems or sub-systems of the dental computing environment 1200 may access or use the patient's dental ridgeline information generated by the methods and apparatuses described herein. For example, the doctor system 1220 may include treatment management modules 1221 and intraoral state capture modules 1222 that may access or use dental ridgeline information. The doctor system 1220 may provide a “doctor facing” interface to the computing environment 1200. The treatment management modules 1221 can perform any operations that enable a doctor or other clinician to manage the treatment of any patient. In some examples, the treatment management modules 1221 may provide a visualization and/or simulation of the patient's dentition with respect to a treatment plan. For example, the doctor system 1220 may include a user interface for the doctor that allows the doctor to manipulate a 3D model and determine dental ridgelines of one or more teeth included in the 3D model. The ridgelines may be used to determine tooth position, such as an initial tooth position that is used with a dental treatment plan to display a predicted final tooth position.

The intraoral state capture modules 1222 can provide images of the patient's dentition to a clinician through the doctor system 1220. The images may be captured through the intraoral scanning system 1210 and may also include images of a simulation of tooth movement based on a treatment plan.

In some examples, the treatment management modules 1221 can enable the doctor to modify or revise a treatment plan, particularly when images provided by the intraoral state capture modules 1222 are used to determine one or more dental ridgelines that can identify a tooth's position (initial position). The tooth's position may be used to predict the tooth's final position based on the treatment plan. The doctor system 1220 may include one or more processors configured to execute any feasible non-transitory computer-readable instructions to perform any feasible operations described herein.

Alternatively or additionally, the treatment planning system 1230 may include any of the methods and apparatuses described herein, and/or may determine dental ridgelines from 3D dental models. The treatment planning system 1230 may include scan processing/detailing modules 1231, segmentation modules 1232, staging modules 1233, treatment monitoring modules 1234, treatment planning database(s) 1235, and ridgeline management modules 1236. In general, the treatment planning system 1230 can determine a treatment plan for any feasible patient. The scan processing/detailing modules 1231 can receive or obtain dental scans (such as scans from the intraoral scanning system 1210) and can process the scans to “clean” them by removing scan errors and, in some cases, enhancing details of the scanned image.

The treatment planning system 1230 may include a segmentation system that segments a model into separate components. For example, the treatment planning system 1230 may include a segmentation modules 1232 that can segment a dental model (such as a 3D dental model) into separate parts including separate teeth, gums, jaw bones, and the like. In some cases, the dental models may be based on scan data from the scan processing/detailing modules 1231.

The staging modules 1233 may determine different stages of a treatment plan. Each stage may correspond to a different dental aligner. In some examples, the staging modules 1233 may also determine the final position of the patient's teeth, in accordance with a treatment plan. Thus, the staging modules 1233 can determine some or all of a patient's orthodontic treatment plan. In some examples, the staging modules 1233 can simulate movement of a patient's teeth in accordance with the different stages of the patient's treatment plan.

The treatment monitoring modules 1234 can monitor the progress of an orthodontic treatment plan. In some examples, the treatment monitoring modules 1234 can provide an analysis of progress of treatment plans to a clinician. The orthodontic treatment plans may be stored in the treatment planning database(s) 1235. Although not shown here, the treatment planning system 1230 can include one or more processors configured to execute any feasible non-transitory computer-readable instructions to perform any feasible operations described herein.

The patient system 1240 can include a treatment visualization module 1241 and an intraoral state capture module 1242. In general, the patient system 1240 can provide a “patient facing” interface to the computing environment 1200. The treatment visualization module 1241 can enable the patient to visualize how an orthodontic treatment plan has progressed and also visualize a predicted outcome (e.g., a final position of teeth).

In some examples, the patient system 1240 can capture dentition scans for the treatment visualization module 1241 through the intraoral state capture module 1242. The intraoral state capture module can enable a patient to capture his or her own dentition through the intraoral scanning system 1210. Although not shown here, the patient system 1240 can include one or more processors configured to execute any feasible non-transitory computer-readable instructions to perform any feasible operations described herein.

The ridgeline management modules 1236 can determine ridgelines associated with the scans (3D scans) of one or more teeth. In some cases, the ridgeline management modules 1236 may execute neural networks or execute one or more algorithms to determine any ridgelines associated with any teeth. In some examples, the ridgeline management modules 1236 may execute algorithms for regular shaped teeth and execute one or more neural networks for irregular shaped teeth.

The appliance fabrication system 1250 can include appliance fabrication machinery 1251, processor(s) 1252, memory 1253, and appliance generation modules 1254. In general, the appliance fabrication system 1250 can directly or indirectly fabricate aligners to implement an orthodontic treatment plan. In some examples, the orthodontic treatment plan may be stored in the treatment planning database(s) 1235.

The appliance fabrication machinery 1251 may include any feasible implement or apparatus that can fabricate any suitable dental aligner. The appliance generation modules 1254 may include any non-transitory computer-readable instructions that, when executed by the processor(s) 1252, can direct the appliance fabrication machinery 1251 to produce one or more dental aligners. The memory 1253 may store data or instructions for use by the processor(s) 1252. In some examples, the memory 1253 may temporarily store a treatment plan, dental models, or intraoral scans.

The computer-readable medium 1260 may include some or all of the elements described herein with respect to the dental computing environment 1200. The computer-readable medium 1260 may include non-transitory computer-readable instructions that, when executed by a processor, can provide the functionality of any device, machine, or module described herein.

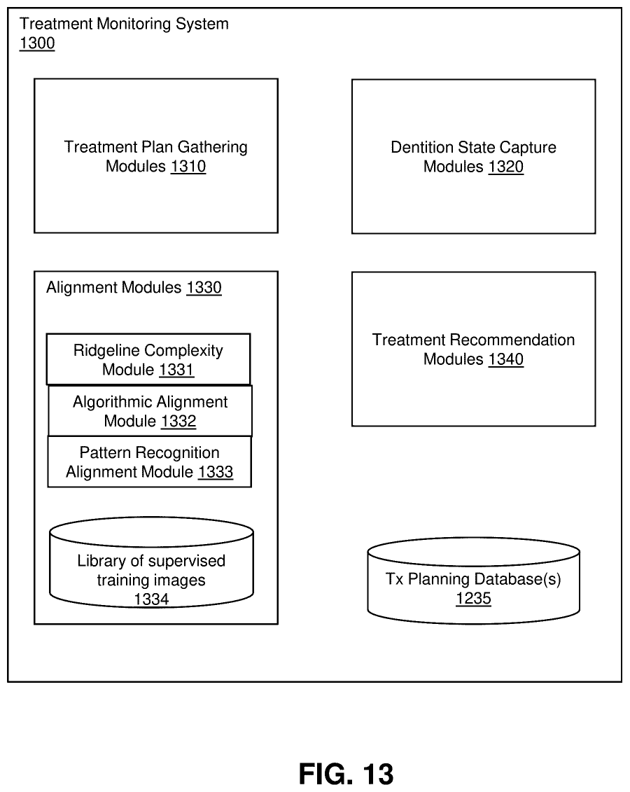

FIG. 13 is a block diagram of one example of treatment monitoring modules 1300. The treatment monitoring modules 1300 may be an example of the treatment monitoring modules 1234 of FIG. 12. The treatment monitoring modules 1300 may include a treatment plan gathering module 1310, a dentition state capture module 1320, an alignment module 1330, and a treatment recommendation module 1340. In general, the treatment monitoring system 1300 can enable a clinician to monitor a patient's progress with respect to a treatment plan.

The treatment plan gathering module 1310 can retrieve a patient's treatment plan. In some cases, the patient's treatment plan may be stored in, and retrieved from, the treatment planning database(s) 1235 of FIG. 12. The dentition state capture module 1320 can capture or obtain any feasible images of the patient's dentition. In some examples, the dentition state capture module 1320 can execute or perform any operations described with respect to the intraoral scanning system 1210.

The alignment module 1330 can align elements or objects from the treatment plan with elements or objects from an image of the patient's dentition. For example, the alignment module 1330 can align or match teeth (teeth positions) that have been described by a treatment plan to teeth that have been captured through an image scan.

In some examples, the alignment module 1330 may include ridgeline complexity module 1331, an algorithmic alignment module 1332, a pattern recognition alignment module 1333, and a library of supervised training images 1334. As described above with respect to FIG. 10, ridgelines for a tooth may be determined at least by an algorithmic approach, or with a neural network-based approach. The neural network-based approach may use 3D scan data to identify ridgelines by recognizing patterns of ridgelines through trained neural networks.

The ridgeline complexity module 1331 determines if a tooth has an irregular (complex) or non-irregular (simple) shape. Based on the determined shape, the alignment module 1330 may use an algorithmic or a pattern recognition alignment module to determine dental ridgelines.

The algorithmic alignment module 1332 may determine dental ridgelines using conventional algorithmic approaches. The pattern recognition alignment module 1333 can determine dental ridgelines through the execution of one or more trained neural networks. The neural networks may be trained with supervised training images. In some examples, the alignment module 1330 may include the library of supervised training images 1334 used to train any neural network.

The treatment recommendation module 1340 can determine whether any changes in the treatment plan may be needed to achieve a particular final position of teeth. For example, any misalignment between the position of teeth in an image scan and the position of teeth (such as a misalignment greater than a predetermined amount) described by the treatment plan may indicate an undesirable outcome. The treatment recommendation module 1340 can recommend a change in the treatment plan to address any potential undesirable outcome. On the other hand, if there is a misalignment less than a predetermined amount, then the treatment recommendation module 1340 may not suggest any changes in the treatment plan.

All or some of the components described above, including in FIG. 2, may be incorporated in the system shown in FIGS. 12 and/or 13. Similarly, any of the components (e.g., modules, engines, etc.) described in FIGS. 12 and 13 may be incorporated into a system such as that shown in FIG. 2.

The embodiments described herein may be used to determine the dental ridgeline of a patient's dentition. The dental ridgelines may be used to determine a tooth's position, which in turn can be used to determine the tooth's final position in response to a dental treatment plan that may be used for virtual oral diagnostics and visualizations throughout an orthodontic treatment process.

As described herein, a system, apparatus, and/or method is described to identify dental ridgelines on teeth, even irregularly shaped teeth that may have ridgelines that are not casily identified with conventional (algorithmic) approaches. The systems, apparatus, and/or methods may include the training and execution of a neural network to recognize and/or identify dental ridgelines. The neural networks may be trained to recognize and/or identify dental ridgelines on irregularly shaped, chipped, broken, etc., teeth which previously had ridgelines that could not be identified.

Some advantages include a more accurate determination and visualization of a final position of teeth responding to a proposed treatment plan. In some examples, both conventional (algorithmic) and neural-network based approaches may be used to determine dental ridgelines. In some case the approach is selected based on a tooth's shape. Using multiple approaches to determine dental ridgelines may improve the accuracy of any predicted final tooth position.

In general, the trained neural network may determine areas of probabilities that may be associated with one or more ridgelines. By connecting the areas of highest (relatively highest) probabilities, a ridgeline may be determined. The neural networks may be trained to identify or locate the regions on a tooth that are the most probable for being a ridgeline. INTELLIGENT STAGING AND DIGITAL TREATMENT PLANNING

Any of the methods and systems described herein may also or alternatively relate to optimizing computerized staging patterns that are used as the basis of digital treatment planning on electronic devices. The methods and systems described herein may be used to identify certain tooth movements in a digital treatment plan that may be in excess of a desired tooth movement, thereby allowing for adjustment of the digital treatment plan. Such techniques may allow for more flexibility and customized design in treatment planning.

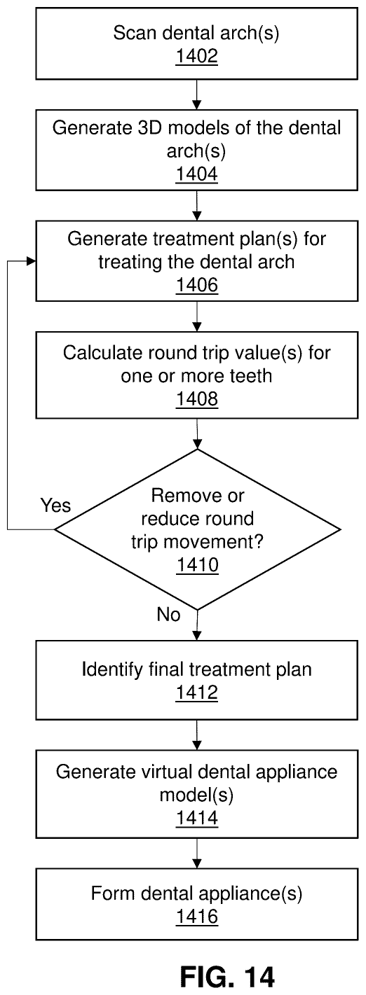

FIG. 14 is a flowchart indicating an example process for treatment planning for treating a dental arch. At step 1402, dental arch(es) are scanned (and/or otherwise imaged) to acquire dimensional information for the dental arch(es). The dental arch(es) may include one or both of a patient's dental arches of the upper and lower jaws. In some cases, the scanning is performed using an intraoral scanner specialized to obtain images inside a patient's mouth. In some examples, the intraoral scanner is configured to collect images of external and/or internal features of the oral cavity. For example, the intraoral scanner may be configured to obtain external surfaces of the patient's teeth and soft tissue and/or tooth roots and unerupted teeth under the soft tissue. In some cases, the intraoral scanner may be configured to use one or more modes of electromagnetic radiation (e.g., visible, infrared, ultraviolet and/or x-rays).

At step 1404, one or more three-dimensional (3D) models of the dental arch(es) are generated based in the scan of the dental arch. For example, the scan data may be processed to form a virtual 3D mesh of the dental arch. A 3D model of a patient's current teeth may be used as a basis to generate one or more target 3D models representing the patient's teeth in one or more desired (e.g., straightened) arrangements. Such target teeth arrangements may be determined based on one or more goals of the treatment plan. Treatment goals may be based on a priority of correcting different malocclusions (e.g., overbite, underbite, reduce tooth crowding, reduce gaps between teeth, improve bite and/or straighten crooked teeth), duration of treatment and/or types and extent of dental procedures (e.g., tooth extraction and/or implants).

At step 1406, one or more treatment plans for treating the dental arch are generated. This may involve calculating transformations that will move the teeth along tooth movement paths from their current (e.g., initial) positions to the one or more target (e.g., final) positions. The treatment plan may include moving the teeth through a sequence of intermediate stages to accomplish the target arrangement of the teeth. Each of the intermediate stages may be represented with an associated intermediate 3D model. Each stage of the treatment plan may be associated with a dental device (e.g., aligner) that the patient wears on their teeth. For example, a first aligner may be configured to move the patient's teeth from a first arrangement to a second arrangement, a second aligner may be configured to move the patient's teeth from the second arrangement to a third arrangement, and so on until a target arrangement of the teeth is accomplished.

Treatment planning may be at least partially automated. That is, a program stored within a computing device may be configured to analyze the current and target tooth arrangements, and automatically create a route for each to move from its current position to its target position, including the intermediate tooth arrangements. In doing such, the program may be configured to coordinate the movement of the teeth such that the simplest method of moving teeth is utilized based upon several factors (e.g., complexity of movement required and/or obstructions from other teeth). The teeth may be scheduled to move according to various movement patterns at different times during the treatment duration. Examples of such tooth movement patterns and associated optimization techniques are described in U.S. Pat. No. 8,038,444, which is incorporated by reference herein in its entirety.

At step 1408, one or more round trip values for one or more corresponding teeth are calculated. A “round-trip” is a tooth movement pattern where a tooth moves in one direction (e.g., forward) and then moves in opposite direction (e.g., backward) for some distance. Round tripping may be necessary for some treatments. Some treatments may not be possible without it. For example, a first tooth may need to be moved out of the path of a second tooth, and once the second tooth has sufficiently moved, the first tooth can be moved back toward its previous position. Even though the tooth is moved in a direction towards its previous position, it may not move precisely back to its previous position, but may still be considered a round trip.

Although round trip movements may be useful in certain situations in moving teeth to their target positions, round trip movements may not always be necessary or ideal. For example, moving a tooth in one direction and then back in the opposite direction may be unnecessary and may lead to an increase in the treatment duration. In some cases, a round trip may be included as part of a treatment plan in error by treatment plan software and/or by a manual staging modification. The methods and systems described herein address problems associated with round trip movements by evaluating round trip movements of one or more teeth. For example, a round trip value, which is a measure of the extent to which a tooth moves back toward its previous position, may be calculated. If the round trip value is high, this may be an indication that the tooth movement may be too extensive and/or may be unnecessary. Tooth movements that have high round trip values may be flagged and/or evaluated to determine whether to reduce the tooth movement or eliminate the round trip movement all together. Additionally or alternatively, the stage of a treatment plan that contributes most to the round trip (e.g., associated with the most movement) of a tooth may be calculated and identified as a “peak stage”.

At step 1410, a decision is made whether to remove the one or more identified round trips, or to reduce the tooth movement in the one or more round trips. In some examples, this decision may be made automatically by a program stored within a computing device. The such an automated decision may be based on whether the identified round trip(s) meet pre-defined criteria, for example, whether a round trip value is above a threshold value and/or whether a peak stage corresponds to one or more particular stages of the treatment plan. In some examples, the threshold value(s) and/or particular stage(s) may be adjustable. In some examples, the program is configured to manually receive instructions from a user (e.g., dental practitioner) to remove one or more identified round trips, or to reduce the tooth movement in the one or more round trips. For example, the program may be configured to display the round trip value(s) and/or peak stage(s) to a user via a user interface, and to receive instructions from the user.

If the decision is to remove or modify the one or more round trips (“Yes”), one or more new treatment plans may be generated based on the adjusted parameters (return to step 1406). If the decision is not to remove or modify the one or more round trips (“No”), the process can proceed to the next step 1412.

At step 1412, a final treatment plan is identified. In some cases, the final treatment plan is chosen from multiple treatment plans generated based on various treatment goals. The final treatment plan may be decided based on consultation with the patient as to which treatment goals the patient feels are most important. Once the final treatment plan has been identified, the virtual 3D models of the target (e.g., final) and intermediate teeth arrangements may also be identified.

At step 1414, one or more virtual dental appliance models are generated based on the final treatment plan. The virtual dental appliances (e.g., virtual aligners) may be 3D digital representations of dental appliances for implementing the final treatment plan. The virtual dental appliances may be generated based on simulating forces applied to the intermediate virtual teeth models. For example, a first virtual dental appliance may be shaped to apply virtual forces on a first intermediate virtual teeth model to move the teeth toward a second subsequent intermediate virtual teeth model, and a second virtual dental appliance may be shaped to apply virtual forces on the second intermediate virtual teeth model to move the teeth toward a third subsequent intermediate virtual teeth model. This process can be repeated for each stage of the treatment plan until a sequence of virtual dental appliances for implementing the full treatment plan are generated.

At step 1416, one or more dental appliances are generated based on the one or more virtual dental appliances. The dental appliances may be a series of dental appliances, where each dental appliance of the series is configured to implement a corresponding stage of the treatment plan. In some cases, the dental appliances include aligners (e.g., clear aligners) that are made of polymer material and that are shaped to removably fit on the patient's dental arch. An aligner may include tooth receiving cavities that are shaped to receive one or more teeth of a dental arch and that include walls (e.g., buccal, facial and/or occlusal walls) that are slightly offset with respect to surfaces of the patient's teeth so as to resiliently apply repositioning forces on the teeth. In some examples, an aligner is shaped to engage with one or more dental attachments that are attached to the patient's teeth. For example, an aligner may include an attachment cavity that is arranged to receive a dental attachment, where the attachment cavity has walls that apply repositioning forces on a surface (e.g., flat surface) of the dental attachment. This force can be transferred to the tooth on which the dental attachment is attached and in a direction in accordance with the stage of the treatment plan. In some examples, one or more of the dental appliances is configured to apply outward forces on lingual surfaces of one or more of the patient's molars, thereby providing an expansion force on the patient's palate.

In some examples, at least a portion of a dental appliance may be made of one or more polymer materials. In some examples, the polymer material may be a thermoplastic material. In some examples, the polymer material may be a thermoset material. In some examples, a dental appliance may be made of a combination of thermoplastic and thermoset materials.

The dental appliances may be fabricated using any of a number of manufacturing techniques. In some examples, at least a portion of a dental appliance is fabricated using a 3D printing process. In some examples, at least a portion of a dental appliance is fabricated using a molding process. In some examples, a dental appliance may be fabricated using a combination of 3D printing and molding processes.

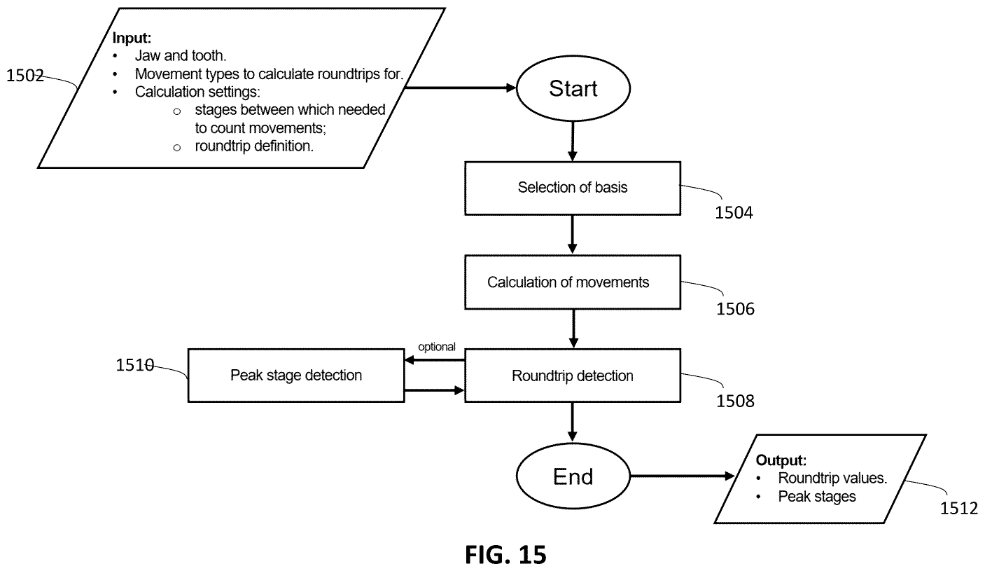

FIG. 15 is a flowchart indicating an example process for evaluating round trips in a treatment plan using a computing device. At step 1502, the computing device receives treatment plan parameters as input. Treatment plan parameters may include jaw and teeth positional data for the initial, final and intermediate stages of one or more treatment plans. Jaw and teeth positional data may include relative tooth positions of the dental arch of one or both of the upper and lower jaws (e.g., virtual 3D models of the upper and/or lower dental arches). The input may also include tooth movement types, which are used to calculate tooth movements at step 1506. Tooth movement types may include specific translations and/or rotations of the tooth in three-dimensional space, which is described further with reference to step 204. The input may also include calculation settings, which may include the treatment plan stages (between which the tooth movements are counted) and definition of a round trip, which are conditions for calculating round trips at step 208.

At step 1504, the basis for each tooth movement direction and origin is selected. In some examples, the origin may be one of the following: crown center, root center, root apex, or tooth tip. Movements may include translations (e.g., in three directions/dimensions) and/or rotations. Table 1 indicates example bases according to movement directions and origins.

TABLE 1

| Movement direction | Basis |
|---|---|
| Buccal-lingual translations, | Basis eₓ, e_{y}, e_{z} calculated dependently to the |
| Mesial-distal translations | jaw arch at the last treatment stage: |
| | e_{z} is a vector with coordinates (0, 0, 1). |
| | e_{y} is a tangent to the final jaw arch in a point |
| | of the arch closest to the tooth crown center. |
| | eₓ is a vector product of e_{z} and e_{y}. |
| Extrusion-intrusion | Depends on movement origin: |
| translations | if the movement origin is crown or root |
| | center the tooth basis is used, |
| | if the movement origin is tooth tip point, |
| | then basis defined in calculation settings is |
| | used (jaw pair or tooth basis). |
| Mesial-distal rotation, | Tooth basis |
| Mesial-distal angulation, | |
| Proclination, Retroclination | |

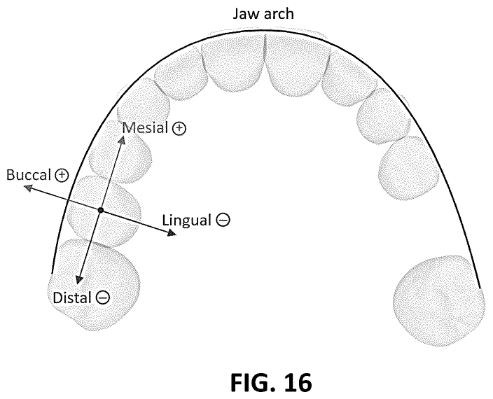

FIG. 16 shows a jaw arch illustrating examples of translational movement along a buccal-lingual axis and along a mesial-distal axis of a tooth. In this example, translation in the buccal direction along the buccal-lingual axis is given a positive value, and translation in the lingual direction along the buccal-lingual axis is given a negative value. Also in this example, translation in the mesial direction along the mesial-distal axis is given a positive value, and translation in the distal direction along the mesial-distal axis is given a negative value.

Buccal-lingual and mesial-distal movements can be referred to as horizontal movements. To create a basis for horizontal movements, the jaw arch should be built previously.

Returning to the flowchart of FIG. 15, at step 1506, tooth movements between treatment stages (in the basis selected in step 1504) are calculated. These calculated movements may be saved in an array. The treatment stages may be divided into two groups: key stages and all other stages. Key stages are stages at which a tooth has a predetermined position. That is, the tooth as an explicitly specified position in a key stage. All other stages are stages that are not key stages, at which a tooth position is calculated as a linear interpolation between adjacent key stages (previous and next nearest key stage). Since, according to some examples, a tooth's position at all stages may be accessed identically, there may be no differences between key and non-key stages. A set of integer pairs may be created, which defines the stages between which movements are calculated. The set of integer pairs may be formed according to information defined in the calculation settings (see step 1502). Table 2 provides exemplary types of stages.

TABLE 2

| Stages | Description |
|---|---|
| From start to keys | The movements are calculated between the |
| | initial and intermediate key treatment stages. |
| Between keys | The movements are calculated only between |
| | intermediate key treatment stages. |
| Between all stages | The movements are calculated between all |
| | treatment stages. |

At step 1508, one or more round trips in the treatment plan may be detected based on the tooth movements calculated at step 1506. Round trips may be detected based on two different types of calculations: 1) unplanned positive movement, and/or 2) minimal total direction movement. Which round trip type of calculation is used, and if one or both types of round trip calculations are used, may be specified in calculation settings (step 1502). Only the array with the calculated movements (calculated at step 1506) may be needed to detect round trips for both types of calculations. The magnitude of a round trip may be calculated and represented as a round trip value. Details of detecting and calculating round trip values using the “unplanned positive movement” and “minimal total direction movement” techniques are described further herein.

At step 1510, the round trip calculation may optionally include determining a peak stage of a treatment plan. The peak stage corresponds to the stage of the treatment plan that contributes most to a round trip value. In some cases, such information may be valuable in determining whether to eliminate or keep a round trip movement and/or to determine what treatment stages should be modified to reduce roundtripping.

At step 1512, one or more round trip values for one or more corresponding teeth are provided as output. In cases where peak stage(s) is/are calculated, one or more peak stages of one or more corresponding teeth may also be provided as output. In some examples, the round trip value(s) and/or peak stage(s) for each tooth may be presented to a user, for example, via a user interface. The user (e.g., dental practitioner) may then choose to eliminate any of the identified round trips or reduce the extent of any identified round trip movements (e.g., thereby reducing the round trip value). The user may base their decision on the round trip values since these values are associated with the extent (e.g., magnitude) of round trip movement of the tooth. Alternatively or additionally, the user may base their decision on the peak stage (if provided). For example, the user may decide to modify the movement of a tooth, or modify the timing of movement of the tooth, based on the round trip value and/or which stage is the peak stage.

In some examples, the system may automatically decide to modify the treatment plan by eliminating one or more identified round trips and/or reducing the extent of one or more identified round trip movements (e.g., thereby reducing the round trip value). Such decision may be made based on the round trip value(s) and/or the peak stage(s). For example, a computer program may include instructions to eliminate, or reduce the effect of, round trip movement of a tooth in a treatment plan if an associated round trip value is above a pre-defined threshold value and/or an associated peak stage corresponds to a pre-defined stage in the treatment plan.

### Unplanned Movement Calculation

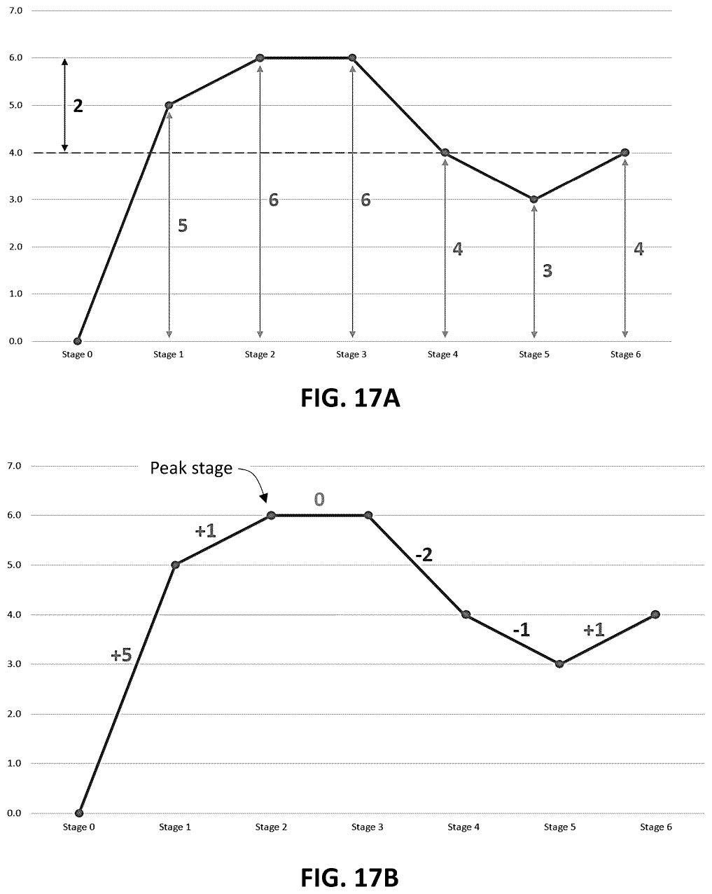

The unplanned movement round trip calculation is applicable only for movements calculated from an initial stage to intermediate keys stages (“From start to keys” in Table 2). A planned movement is a movement from an initial stage to the last tooth key stage (i.e., last key stage value in the calculated movements array). Round trips are calculated as a sum of movements in in the same direction as planned movement that has greater modulo than planned movement. FIG. 17A is an example graph showing how to calculate a round trip value for positive planned movement according to the “unplanned movement” technique. In the graph of FIG. 17A, the x-axis corresponds to the stage of the treatment plan and the y-axis corresponds to distance (e.g., unitless relative distance). In the example of FIG. 17A, the planned movement is 4.0 and round trip value is 2.0, because it contains two positive movements that exceed the planned movement: 1) from 4.0 to 5.0 as a part of movement from stage 0 to stage 1, and 2) whole movement from stage 1 to stage 2. FIG. 17A may be used to calculate positive and negative movements. For example, for a negative movement, the graph of FIG. 17A may be mirrored so that positive values are reflected as negative values.

### Minimal Total Direction Movement Calculation

The minimal total direction movement round trip calculation is applicable for movements calculated between intermediate keys stages (“Between keys” in Table 2) or between all stages (“Between all stages in Table 2). FIG. 17B is an example graph showing how to calculate a round trip value according to the “minimal total direction movement” technique. In the graph of FIG. 17B, the x-axis corresponds to the stage of the treatment plan and the y-axis corresponds to distance (e.g., unitless relative distance). Positive movements are accumulated in a Movement (+) variable. Negative movements are accumulated in a Movement (−) variable. The round trip value is calculated as min (Movement (+),-Movement (−)). According to the “minimal total direction movement” condition, a round trip value will be the same for opposite directions (e.g., buccal and lingual, or mesial and distal). For the example shown in FIG. 17B, the round trip value is calculated as 3.0 because Movement (+) is 5+1+1=7.0 and Movement (−) is −2−1=−3.0.

As shown in FIG. 17B, the peak stage may also be calculated. The peak stage is the first stage that contributes the greatest round trip value. For each stage, the algorithm finds the first nonzero movement before (previous movement) and after (next movement) that stage. If the previous and next movements have different signs, then the “local” round trip around this stage is a minimum of modulus of these movements, otherwise this stage is ignored. The peak stage is the first stage with the greatest “local” round trip. For the example in FIG. 17B, stages 2, 3 and 5 have “local” round trips equal to 1.0, but the peak stage is stage 2.

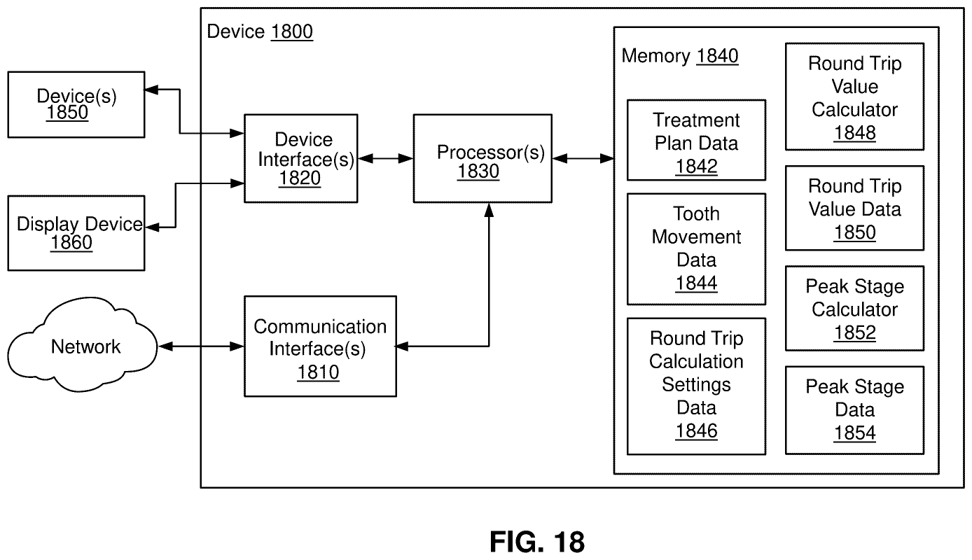

FIG. 18 shows a block diagram of an example system that includes a device 1800 for implementing one or more of the methods described herein. Although described herein as a device, the functionality of the device 1800 may be performed by any feasible apparatus, system, or method. The device 1800 may include one or more communication interfaces 1810, one or more device interfaces 1820, one or more processors 1830, and memory 1840. The device 1800 may be configured to analyze treatment plan parameters for round trip tooth movements and generate output resulting from the analysis. In some examples, the device 1800 is configured to generate one or more treatment plans. In other examples, the device 1800 is configured to receive data associated with a treatment plan that was generated by a separate device (e.g., 1850). The device shown in FIG. 18 may be incorporated or integrated into any system or sub-system for generating a dental treatment and/or dental appliance to perform a treatment, as will be described in greater detail in FIG. 7.

In FIG. 18, the communication interface 1810, which may be coupled to a network and to the processor 1830, may transmit signals to and receive signals from other wired or wireless devices, including remote (e.g., cloud-based) storage devices, cameras, and/or displays. For example, the communication interface 1810 may include wired (e.g., serial, ethernet, or the like) and/or wireless (Bluetooth, Wi-Fi, cellular, or the like) transceivers that may communicate with any other feasible device through any feasible network.

The device interface 1820, which is coupled to the processor 1830, may be used to interface with any feasible input and/or output device. For example, the device interface 1820 may be coupled to an interface with one or more devices (e.g., 1850) that are configured to generate treatment plans. In some examples, the device interface 1820 may be coupled to an interface with a display device 1860. Through a user interface of the display device 1860, the processor 1830 may display and/or receive information related to the round trip calculations. In some examples, the processor 1830 is configured to display virtual models (e.g., 3D models) of dental arches. The processor 1830 may be configured to receive feedback information from the display device 1860 and/or other devices (e.g., 1850).

In some examples, the device(s) 1850 and/or the display device 1860 may be an integral part of the device 1800. In other words, the device(s) 1850 and/or the display device 1860 may share a common housing or enclosure and/or share one or more processors (e.g., 1830). In some examples, the device 1800 may be a portable electronic device (e.g., cell phone, a tablet computer, a laptop computer) or a desktop computer that includes at least these elements.

The processor(s) 1830, which is coupled to the memory 1840, may be any one or more suitable processors capable of executing scripts or instructions of one or more software programs stored in the device 1800 (such as within memory 1840).

The memory 1840 may include a non-transitory computer-readable storage medium (e.g., one or more nonvolatile memory elements, such as EPROM, EEPROM, Flash memory, a hard drive, etc.).

The memory 1840 may include treatment plan data 1842 for one or both of a patient's jaws. The treatment plan data 1842 may include 3D positional data for initial, final and intermediate treatment stages. The treatment plan data 1842 may include segmented data where each of the teeth is identified as a virtual object and numbered according to tooth number. In some cases, the treatment plan data 1842 may include virtual 3D models of the initial, final and intermediate tooth arrangements.

The memory 1840 may include tooth movement data 1844 for each tooth between each of the stages. The tooth movement data 1844 may calculated via the processor 1830 (e.g., step 206) and stored as an array arranged according to stages of the treatment plan.

The memory 1840 may include round trip calculation settings data 1846 used to calculate the tooth movement data 1844. The round trip calculation settings data 1846 may include the stages between which the tooth movements are to be calculated (See, e.g., Table 2). The round trip calculation settings data 1846 may also include the settings for the round trip calculation. Settings may include the conditions for using different types of round trip calculation (e.g., “unplanned positive movement” calculation and/or “minimal total direction movement” calculation). The settings may also include one or more threshold values for the round trip value and/or one or more peak stage settings. In some cases, the settings may be adjustable, for example, by the user (e.g., dental practitioner).

The processor 1830 may be configured to execute a round trip value calculator module 1848 stored on the memory 1840 to calculate one or more round trip values for one or more corresponding teeth. The resulting round trip value data 1850 may be stored in the memory 1840.

The processor 1830 may optionally be configured to execute a peak stage calculator module 1852 stored on the memory 1840 to calculate one or more peak stages for one or more corresponding teeth. The resulting peak stages data 1854 may be stored in the memory 1840.

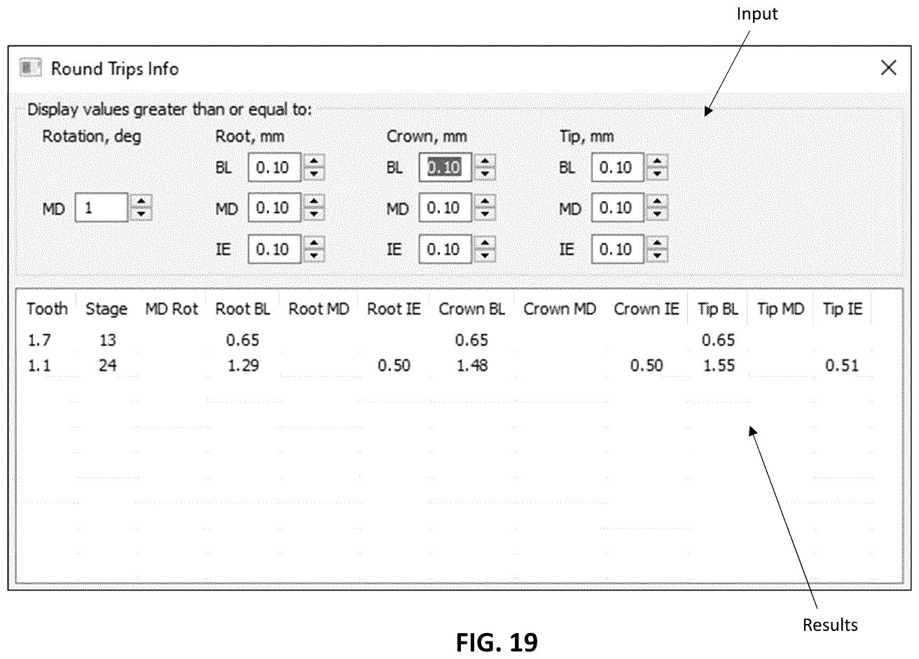

FIG. 19 shows an example user interface for digital treatment planning. In this example, the user interface includes an input area where a user can enter threshold values (values greater than or equal to) for calculating roundtrips of the teeth. For example, the user may enter threshold values for mesial-distal (MD) rotation (degrees), buccal-lingual (BL) movement of the root (mm), MD movement of the root (mm), intrusion-extrusion (IE) movement of the root (mm), BL movement of the crown (mm), MD movement of the crown (mm), IE movement of the crown (mm), BL movement of the tip (mm), MD movement of the tip (mm), and IE movement of the tip (mm). In this example, a results area of the user interface displays results of the roundtrip calculations. The results may identify teeth that are identified as having a roundtrip according to the threshold criteria. Results may include the tooth number (Tooth), the stage of the treatment plan (Stage), MD rotation (degrees), Root BL (mm), Root MD (mm), Root IE (mm), Crown BL (mm), Crown MD (mm), Crown IE (mm), Tip BL (mm), Tip MD (mm), and Tip IE (mm).

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

For example, FIG. 12, discussed above, illustrates an example of a system that may include the methods and apparatuses described herein for estimating and/or modifying tooth round trip movements during treatment. For example, the methods and apparatuses (systems, devices, etc., including software, hardware and/or firmware) described herein for estimating and/or modifying tooth round trip movements may be used at one or more parts of a dental computing environment, including as part of an intraoral scanning system, doctor system, treatment planning (e.g., technician) system, patient system, and/or fabrication system. In particular, these methods and apparatuses may be used as part of the treatment planning by the technician (e.g., treatment planning system or subsystem), and/or the doctor system or subsystem.

Returning now to FIG. 12, as discussed above, this diagram illustrates a variation of a computing environment 1200 that may generate one or more orthodontic treatment plans specific to a patient, and fabricate dental appliances that may accomplish the treatment plan to treat a patient, under the direction of a dental professional. In general, any of these systems may include or reference the round tripping modules for estimating round trip movements and modifying the treatment plan (e.g., tooth movements) based on the round-trip estimations, as described herein. Thus, any of the systems described herein may incorporate all or some of the features described in reference to FIG. 18, above. In FIG. 12, the doctor system 1220 may also display, access or include the round trip estimation(s) as described herein.

In some examples, the treatment management module 1221 can enable the doctor to modify or revise a treatment plan, particularly when images provided by the intraoral state capture module 1222 indicate that the movement of the patient's teeth may not be according to the treatment plan. In some examples the treatment management module 1221 may access or include the round trip module(s), including displaying and/or modifying tooth movements based on round-trip estimations performed as described herein. The doctor system 1220 may include one or more processors configured to execute any feasible non-transitory computer-readable instructions to perform any feasible operations described herein.

Alternatively or additionally, the treatment planning system 1230 may include any of the methods and apparatuses described herein. The treatment planning system 1230 may include scan processing/detailing module 1231, segmentation module 1232, staging module 1233, treatment monitoring module 1234, and treatment planning database(s) 1235. The treatment planning system 1230 may generally include the round trip modules 1236; in the example shown in FIG. 12, the round-trip modules may be part of the staging module(s) 1233. Alternatively, the round tripping module(s) may be separate from the staging module(s) and/or may be part of one or more other modules. In general, the treatment planning system 1230 can determine a treatment plan for any feasible patient. The scan processing/detailing module 1231 can receive or obtain dental scans (such as scans from the intraoral scanning system 1210) and can process the scans to “clean” them by removing scan errors and, in some cases, enhancing details of the scanned image. The treatment planning system 1230 may perform segmentation. For example, a treatment planning system may include a segmentation module 1232 that can segment a dental model into separate parts including separate teeth, gums, jaw bones, and the like. In some cases, the dental models may be based on scan data from the scan processing/detailing module 1231.

As discussed above, the staging module 1233 may determine different stages of a treatment plan. Each stage may correspond to a different dental aligner. The staging module 1233 may also determine the final position of the patient's teeth, in accordance with a treatment plan. Thus, the staging module 1233 can determine some or all of a patient's orthodontic treatment plan. In some examples, the staging module 1233 can simulate movement of a patient's teeth in accordance with the different stages of the patient's treatment plan. As mentioned, the staging module may include and/or may access the round tripping module(s) 1236.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system comprising:
one or more processors;
memory coupled to the one or more processors, wherein the memory includes computer-program instructions that, when executed by the one or more processors, cause the device to perform operations comprising:
acquiring three-dimensional (3D) scan data of a patient's dentition;
converting the 3D scan data into a 3D model of the patient's dentition;
segmenting the 3D model of the patient's dentition into individual teeth;
identifying patterns corresponding to two or more ridgeline point locations for each individual tooth in the 3D model of the patient's dentition; and
determining a ridgeline for each individual tooth in the patient's dentition based on identified patterns corresponding to the two or more ridgeline point locations.

2. The system of claim 1, wherein identifying the patterns comprises evaluating the 3D model against a model trained to identify ridgeline point locations on a training dataset of 3D models.

3. The system of claim 1, wherein identifying the patterns comprises evaluating the 3D model against a machine learning model trained to identify ridgeline point locations on a training dataset of 3D models.

4. The system of claim 1, wherein identifying the patterns comprises evaluating the 3D model against a machine learning model trained to identify ridgeline point locations on a training dataset of 3D models, and wherein the machine learning model is trained using a neural network.

5. The system of claim 1, wherein the identified patterns correspond to probabilities of the two ridgeline point locations.

6. The system of claim 1, wherein the computer-program instructions are further configured to perform operations comprising:
obtaining a training dataset of 3D models;
training a neural network to recognize ridgeline point locations on the training dataset of 3D models;
outputting a model trained to identify the ridgeline point locations on the training dataset of 3D models.

7. The system of claim 1, wherein the computer-program instructions are further configured to perform operations comprising:
obtaining a training dataset of 3D models;
training a neural network to recognize ridgeline point locations on the training dataset of 3D models;
outputting a model trained to identify the ridgeline point locations on the training dataset of 3D models;
wherein identifying the patterns comprises evaluating the 3D model against the model trained to identify ridgeline point locations on the training dataset of 3D models.

8. The system of claim 1, wherein the computer-program instructions are further configured to perform operations comprising:
obtaining a training dataset of 3D models;
training a neural network to recognize ridgeline point locations on the training dataset of 3D models;
outputting a model trained to identify the ridgeline point locations on the training dataset of 3D models;
wherein identifying the patterns comprises evaluating the 3D model against the model trained to identify ridgeline point locations on the training dataset of 3D models.

9. The system of claim 1, wherein the two or more ridgeline point locations include mesial, middle, and distal point locations of a ridgeline.

10. The system of claim 1, wherein the neural network includes a classifier to determine probabilities of the two or more ridgeline point locations from the 3D model.

11. The system of claim 1, wherein the neural network includes a classifier to determine probabilities of teeth and gingiva locations for the 3D model.

12. The system of claim 1, wherein training for the neural network includes supervised training with 3D models labeled with tooth ridgelines.

13. The method of claim 1, further comprising determining a treatment plan based at least in part on the determined ridgelines of each individual tooth in the patient's dentition.

14. The method of claim 13, wherein determining the treatment plan includes determining a final position of each tooth in the patient's dentition.

15. A system comprising:
one or more processors;
memory coupled to the one or more processors, wherein the memory includes computer-program instructions that, when executed by the one or more processors, cause the device to perform operations comprising:
converting a patient's 3D dental scan data into a 3D model of the patient's dentition;
segmenting the 3D model of the patient's dentition into individual teeth;
determining whether one or more of the individual teeth include irregular shape characteristics; and
determining a ridgeline for each individual tooth in the patient's dentition based on whether one or more of the individual teeth include irregular shape characteristics.

16. A system comprising:
one or more processors;
memory coupled to the one or more processors, wherein the memory includes computer-program instructions that, when executed by the one or more processors, cause the device to perform operations comprising:
determining a jaw arch from a model of a patient's upper and/or lower jaw;
calculating a roundtrip value from movements relative to the jaw arch or on a tooth basis for one or more teeth of an orthodontic treatment plan comprising a plurality of different stages for moving the patient's teeth, wherein calculating the roundtrip value is performed between all of the stages, between keys of the orthodontic treatment plan, or from start to the keys of the orthodontic treatment plan, further wherein calculating the roundtrip value comprises calculating one or more of: an unplanned movement and/or a minimal total direction movement; and
adjusting the orthodontic treatment plan based on the roundtrip value.

17. The system of claim 16, wherein calculating the roundtrip value comprises calculating buccal-lingual translations and mesial-distal translations relative to the jaw arch.

18. The system of claim 16, wherein calculating the roundtrip value comprises calculating a roundtrip value for extrusion-intrusion movements of the one or more teeth relative to an origin of the one or more teeth.

19. The system of claim 18, wherein the origin is a crown center, root center, root apex, or tooth tip of the one or more teeth.

20. The system of claim 16, wherein calculating the roundtrip value further comprises calculating a roundtrip value for rotation, proclination, or retroclination movements of the one or more teeth on a tooth basis of the one or more teeth.

21. The system of claim 16, wherein the computer-program instructions are further configured to perform operations comprising specifying if the roundtrip value comprises an unplanned movement, or a minimal total direction movement, or both.

22. The system of claim 16, wherein calculating the roundtrip value comprises generating an array with calculated roundtrip values associated with movements for the one or more teeth.

23. The system of claim 16, wherein the computer-program instructions are further configured to perform operations comprising determining which stage contributes the most to the roundtrip value based on which stage is associated with the most movement, and wherein adjusting the orthodontic treatment plan comprises adjusting based on the roundtrip value and on the stage contributing the most to the roundtrip value.

24. The system of claim 16, wherein the computer-program instructions are further configured to perform operations comprising forming one or more dental aligners based on the adjusted orthodontic treatment plan.

25. The system of claim 16, wherein adjusting the orthodontic treatment plan comprises iteratively modifying the orthodontic treatment plan to reduce the roundtrip value.

26. A system comprising:
one or more processors;
memory coupled to the one or more processors, wherein the memory includes computer-program instructions that, when executed by the one or more processors, cause the device to perform operations comprising:
determining a jaw arch to form a coordinate system from a model of a patient's upper and/or lower jaw;
calculating a roundtrip value from movements relative to the jaw arch or on a tooth basis for one or more teeth of an orthodontic treatment plan comprising a plurality of different stages for moving the patient's teeth, wherein calculating the roundtrip value is are performed between all of the stages, between keys of the orthodontic treatment plan, or from start to the keys of the orthodontic treatment plan, further wherein calculating the roundtrip value comprises calculating one or more of: an unplanned movement and/or a minimal total direction movement;
determining which stage of the plurality of different stages of the orthodontic treatment plan contributes the most to the roundtrip value; and
adjusting the orthodontic treatment plan based on the roundtrip value and on the stage that contributes the most to the roundtrip value.
